SEQUENCE LISTING (27 sequences)

SEQ ID NO: 1 - protein, 10 aa, unidentified
  REGION 1..10: note = Description of Unknown: Virion protein 1 polypeptide
  MOD_RES 3..4: note = Any amino acid or absent
  MOD_RES 6: note = Any amino acid or absent
  MOD_RES 9: note = Any amino acid or absent
LAXXDXTKXA

SEQ ID NO: 2 - protein, 10 aa, unidentified
  REGION 1..10: note = Description of Unknown: Virion protein 1 polypeptide
  MOD_RES 3: note = Any amino acid or absent
  MOD_RES 5: note = Any amino acid or absent
  MOD_RES 8..9: note = Any amino acid or absent
LAXDXTKXXA

SEQ ID NO: 3 - protein, 10 aa, unidentified
  REGION 1..10: note = Description of Unknown: Virion protein 1 polypeptide
LADQDYTKTA

SEQ ID NO: 4 - protein, 621 aa, Adeno-associated virus type 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 5 - protein, 536 aa, Adeno-associated virus type 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 6 - protein, 397 aa, Adeno-associated virus type 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 7 - protein, 312 aa, Adeno-associated virus type 2
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 8 - DNA, 1866 bp, Adeno-associated virus type 2
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataa

SEQ ID NO: 9 - protein, 756 aa, unidentified
  REGION 1..756: note = Description of Unknown: AAV9Retro VP1 polypeptide sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLADQDYTKTALADQDYTKTAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 746 aa, unidentified
  REGION 1..746: note = Description of Unknown: AAV9 VP1 polypeptide sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLADQDYTKTAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 609 aa, unidentified
  REGION 1..609: note = Description of Unknown: AAV9Retro VP2 polypeptide sequence
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLADQDYTKTAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 599 aa, unidentified
  REGION 1..599: note = Description of Unknown: AAV9 VP2 polypeptide sequence
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 544 aa, unidentified
  REGION 1..544: note = Description of Unknown: AAV9Retro VP3 polypeptide sequence
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLADQDYTKTAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 534 aa, unidentified
  REGION 1..534: note = Description of Unknown: AAV9 VP3 polypeptide sequence
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 748 aa, unidentified
  REGION 1..748: note = Description of Unknown: AAV10Retro VP1 polypeptide sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 16 - protein, 738 aa, unidentified
  REGION 1..738: note = Description of Unknown: AAV10 VP1 polypeptide sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 17 - protein, 591 aa, unidentified
  REGION 1..591: note = Description of Unknown: AAV10Retro VP2 polypeptide sequence
TGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 18 - protein, 581 aa, unidentified
  REGION 1..581: note = Description of Unknown: AAV10 VP2 polypeptide sequence
TGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 19 - protein, 545 aa, unidentified
  REGION 1..545: note = Description of Unknown: AAV10Retro VP3 polypeptide sequence
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 20 - protein, 535 aa, unidentified
  REGION 1..535: note = Description of Unknown: AAV10 VP3 polypeptide sequence
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 21 - DNA, 30 bp, unidentified
  misc_feature 1..30: note = Description of Unknown: AAV9Retro Cap polynucleotide
  source 1..30: mol_type = genomic DNA
ctagcagaccaagactacacaaaaactgct

SEQ ID NO: 22 - DNA, 2241 bp, unidentified
  misc_feature 1..2241: note = Description of Unknown: AAV9Retro Cap polynucleotide sequence
  source 1..2241: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaactagcagaccaagactacacaaaaactgctgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 23 - DNA, 2211 bp, unidentified
  misc_feature 1..2211: note = Description of Unknown: AAV9 Cap polynucleotide sequence
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 24 - DNA, 2200 bp, unidentified
  misc_feature 1..2200: note = Description of Unknown: AAV10Retro Cap polynucleotide sequence
  source 1..2200: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacctagcagaccaagactacacaaaaactgctgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttatt

SEQ ID NO: 25 - DNA, 2170 bp, unidentified
  misc_feature 1..2170: note = Description of Unknown: AAV10 Cap polynucleotide sequence
  source 1..2170: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttatt

SEQ ID NO: 26 - DNA, 7360 bp, synthetic construct
  misc_feature 1..7360: note = Description of Artificial Sequence: Synthetic polynucleotide
  source 1..7360: mol_type = other DNA
gtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaactagcagaccaagactacacaaaaactgctgcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgag

SEQ ID NO: 27 - DNA, 7366 bp, synthetic construct
  misc_feature 1..7366: note = Description of Artificial Sequence: Synthetic polynucleotide
  source 1..7366: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacctagcagaccaagactacacaaaaactgctgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaattgcttgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgcc

(19) United States
(12) Patent Application Publication
(10) Pub. No.: US 2023/0346985 A1
(43) Pub. Date: Nov. 2, 2023

(54) ADENO-ASSOCIATED VIRUS VECTORS

(71) Applicant: The Children's Medical Center Corporation, Boston, MA (US)

(72) Inventors: Zhigang HE, Boston, MA (US); Xue YAO, Boston, MA (US); Zicong ZHANG, Boston, MA (US); Benedikt BROMMER, Boston, MA (US)

(73) Assignee: The Children's Medical Center Corporation, Boston, MA (US)

(21) Appl. No.: 18/313,158

(22) Filed: May 5, 2023

Related U.S. Application Data
(63) Continuation of application No. PCT/US2021/058088, filed on Nov. 4, 2021.
(60) Provisional application No. 63/110,631, filed on Nov. 6, 2020.

(51) Int. Cl. C12N 15/86; A61K 49/00

(52) U.S. Cl. CPC A61K 49/0097; C12N 15/86; C12N 2750/14123; C12N 2750/14132; C12N 2750/14142; C12N 2750/14143

(57) ABSTRACT

The present invention features adeno-associated virus (AAV) vectors, compositions thereof, and methods of use thereof for transducing neurons with injured axons.

### CROSS REFERENCE TO RELATED APPLICATIONS

This application is a continuation under 35 U.S.C. §111(a) of PCT International Patent Application No. PCT/US2021/058088, filed Nov. 4, 2021, designating the United States and published in English, which claims priority to and benefit of U.S. Provisional Application No. 63/110,631, filed Nov. 6, 2020, the entire contents of each of which are incorporated by reference herein.

### SEQUENCE LISTING

The present application contains a Sequence Listing which has been submitted electronically in XML format following conversion from the originally filed TXT format.

The content of the electronic XML Sequence Listing, (Date of creation: May 4, 2023; Size: 70,328 bytes; Name: 167705-024002US-Sequence_Listing.xml), and the original TXT format, is herein incorporated by reference in its entirety.

### BACKGROUND OF THE INVENTION

Central Nervous System (CNS) injuries, such as spinal cord injury (SCI), traumatic brain injury (TBI), or stroke, result from traumatic insults that damage the cells around the lesions, as well as axons passing the lesion area. Acute spinal cord injury (SCI), as well as neurodegenerative diseases, cause axonal damage that may result in irreversible loss of function affecting not only movement and sensation, but a variety of autonomic functions, as well. Gene therapies hold promise for the treatment of many neurodegenerative disorders and traumatic injuries in the central nervous system. While progress has been made towards improving methods for transgene delivery, a need exists for improved methods for delivering therapeutic polypeptides to the central nervous system.

### SUMMARY OF THE INVENTION

As described below, the present invention features viral vectors, compositions featuring such vectors, and methods of using them for the treatment of injuries and diseases affecting the central nervous system.

In one aspect, the invention features a method for targeted retrograde infection of an injured neuron. The method involves administering a viral particle or virus-like particle to a subject with the injured neuron, thereby infecting the neuron. The viral particle or virus-like particle contains a virion protein 1 (VP1) polypeptide selected from any one or more of AAV9Retro VP1 and AAV10Retro VP1. The polypeptide contains the following amino acid sequence: LAxxDxTKxA (SEQ ID NO: 1) or LAxDxTKxxA (SEQ ID NO: 2), where X is any amino acid or is absent.

In another aspect, the invention features a method for transduction of a neuron proximal to a site of injury. The method involves contacting the neuron with a viral particle or virus-like particle, thereby retrogradely infecting the neuron. The viral particle or virus-like particle contains a virion protein 1 (VP1) polypeptide selected from any one or more of AAV9Retro VP1 and AAV10Retro VP1. The polypeptide contains the following amino acid sequence: LAxxDxTKxA (SEQ ID NO: 1) or LAxDxTKxxA (SEQ ID NO: 2), where X is any amino acid or is absent.

In another aspect, the invention features a method for treatment of a neuron affected by an injury, disease, or disorder in a subject. The method involves contacting the neuron with an effective amount of a viral particle or virus-like particle. The viral particle or virus-like particle contains a virion protein 1 (VP1) polypeptide selected from any one or more of AAV9Retro VP1 and AAV10Retro VP1. The polypeptide contains the following amino acid sequence: LAxxDxTKxA (SEQ ID NO: 1) or LAxDxTKxxA (SEQ ID NO: 2), where X is any amino acid or is absent.

In any of the above aspects, the disease or disorder is a neurodegenerative disease or disorder.

In any of the above aspects, the neuron is contacted within 7 days of the injury. In any of the above aspects, the neuron is contacted within 3-12 hours of the injury.

In any of the above aspects, the injury is caused by a traumatic insult. In any of the above aspects, the injury is a spinal cord injury or traumatic brain injury.

In any of the above aspects, the neuron contains an axon or cell body proximal to an injury.

In any of the above aspects, the cell body of the neuron is in the gigantocellular reticular nucleus (Gi), the sublaterodorsal tegmental nucleus (SLD), the locus coeruleus (LC), the caudal pontine reticular nucleus (PnC), the pontine reticular formation (PnO), the cortex, the hypothalamic nuclei, or the red nucleus.

In any of the above aspects, the neuron is in a mammalian subject. In some embodiments, the mammal is a rodent or a human.

In any of the above aspects, the viral particle or virus-like particle is administered by intravenous injection.

In another aspect, the invention features an expression vector containing a replication open reading frame from adeno-associated virus serotype 2 (AAV2 Rep), and a capsid open reading frame. The capsid open reading frame is selected from any one or more of AAV9Retro Cap and AAV10Retro Cap. The capsid open reading frame encodes a virion protein 1 (VP1) containing the following amino acid sequence: LAxxDxTKxA (SEQ ID NO: 1) or LAxDxTKxxA (SEQ ID NO: 2), where X is any amino acid or is absent.

In another aspect, the invention features a method for producing a viral particle or virus-like particle. The method involves expressing in a cell or in vitro a replication open reading frame from adeno-associated virus serotype 2 (AAV2 Rep), and a capsid open reading frame. The capsid open reading frame is selected from any one or more of AAV9Retro Cap and AAV10Retro Cap. The capsid open reading frame encodes a virion protein 1 (VP1) containing the following amino acid sequence: LAxxDxTKxA (SEQ ID NO: 1) or LAxDxTKxxA (SEQ ID NO: 2), where X is any amino acid or is absent.

In various embodiments, the method further involves introducing into the cell expressing the AAV2 Rep and the AAV9Retro Cap or AAV10Retro Cap a polynucleotide containing a heterologous polynucleotide sequence flanked by two inverted terminal repeats (ITR). In various embodiments, the ITRs are AAV2 ITRs.

In some embodiments, the viral particle or virus-like particle produced by the method involves a capsid containing the VP1 and the heterologous polynucleotide sequence, and the viral particle or virus-like particle is capable of infecting a target cell. In some embodiments, the target cell is a mammalian cell. In some embodiments, the mammalian cell is a human cell. In some embodiments, the target cell is a nerve cell.

In another aspect, the invention features a viral particle or virus-like particle produced by the method of any of the above aspects.

In another aspect, the invention features a cell containing the expression vector of any one of the above aspects or the viral particle or virus-like particle of any of the above aspects.

In some embodiments, the cell is a bacterial, fungal, insect, or mammalian cell. In some embodiments, the mammalian cell is a human cell. In some embodiments, the cell is a nerve cell.

In another aspect, the invention features a composition containing the expression vector of any of the above aspects or the viral particle or virus-like particle of any of the above aspects.

In some embodiments, the composition contains a pharmaceutically acceptable carrier.

In another aspect, the invention features a kit for transduction of an injured neuron. The kit contains the viral particle or virus-like particle of any of the above aspects. The viral particle or virus-like particle comprises a heterologous polynucleotide comprising a polypeptide-encoding sequence under the control of a promoter, and instructions for the use of the kit in the method of any one of the above aspects.

In another aspect, the invention features a method for imaging an injured neuron. The method involves contacting the neuron with an effective amount of a viral particle or virus-like particle, thereby infecting the neuron and expressing the fluorescent protein in the neuron. The viral particle or virus-like particle contains a virion protein 1 (VP1) polypeptide selected from any one or more of AAV9Retro VP1 and AAV10Retro VP1; where the VP1 polypeptide contains the following amino acid sequence: LAxxDxTKxA (SEQ ID NO: 1) or LAxDxTKxxA (SEQ ID NO: 2), where X is any amino acid or is absent. The viral particle or virus-like particle encapsidates a polynucleotide encoding a fluorescent protein. The method further involves fluorescently imaging the fluorescent protein in the neuron.

In some embodiments, the fluorogenic polypeptide is green fluorescent protein (GFP), or tandem dimer Tomato protein (tdTomato).

In any of the above aspects, the VP1 is AAV9Retro VP1. In any of the above aspects, the VP1 is AAV10Retro VP1. In any of the above aspects, the VP1 contains the following amino acid sequence: LADQDYTKTA (SEQ ID NO: 3). In any of the above aspects, the capsid open reading frame is AAV9Retro Cap. In any of the above aspects, the capsid open reading frame is AAV10Retro Cap.

In any of the above aspects, the virus particle or virus-like particle encapsidates a heterologous polynucleotide sequence. In any of the above aspects, the heterologous polynucleotide sequence contains a polypeptide-encoding sequence under the control of a promoter. In some embodiments, the promoter is a constitutive or inducible promoter. In some embodiments, the promoter is a cytomegalovirus enhancer/chicken beta-actin/Rabbit β-globin (CAG) promoter. In some embodiments, heterologous polynucleotide sequence encodes a polypeptide selected from any one or more of growth factors, fluorescent proteins, phosphatase and tensin homolog (PTEN), suppressor of cytokine signaling 3 (SOCS3), or osteopontin (OPN). In some embodiments, the growth factor is insulin-like growth factor 1 (IGF1).

In any of the above aspects, the neuron is injured by a traumatic insult.

The invention provides vectors, compositions, and methods for transducing neurons with injured axons. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AAV replication polypeptide” is meant one or more proteins or fragments thereof that function in viral replication and that have at least about 85% amino acid sequence identity to one or more of the following polypeptide sequences:

AAV2 Rep78 polypeptide sequence:

MPGFYEIVIKVPSDLDEHLPGISDSFVNWVAEKEWELPPDSDMDLNLIEQ
APLTVAEKLQRDFLTEWRRVSKAPEALFFVQFEKGESYFHMHVLVETTGV
KSMVLGRFLSQIREKLIQRIYRGIEPTLPNWFAVTKTRNGAGGGNKVVDE
CYIPNYLLPKTQPELQWAWTNMEQYLSACLNLTERKRLVAQHLTHVSQTQ
EQNKENQNPNSDAPVIRSKTSARYMELVGWLVDKGITSEKQWIQEDQASY
ISFNAASNSRSQIKAALDNAGKIMSLTKTAPDYLVGQQPVEDISSNRIYK
ILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVR
VDQKCKSSAQIDPTPVIVTSNTNMCAVIDGNSTTFEHQQPLQDRMFKFEL
TRRLDHDFGKVTKQEVKDFFRWAKDHVVEVEHEFYVKKGGAKKRPAPSDA
DISEPKRVRESVAQPSTSDAEASINYADRYQNKCSRHVGMNLMLFPCRQC
ERMNQNSNICFTHGQKDCLECFPVSESQPVSVVKKAYQKLCYIHHIMGKV
PDACTACDLVNVDLDDCIFEQ (SEQ ID NO: 4).

AAV2 Rep68 polypeptide sequence:

MPGFYEIVIKVPSDLDEHLPGISDSFVNWVAEKEWELPPDSDMDLNLIEQ
APLTVAEKLQRDFLTEWRRVSKAPEALFFVQFEKGESYFHMHVLVETTGV
KSMVLGRFLSQIREKLIQRIYRGIEPTLPNWFAVTKTRNGAGGGNKVVDE
CYIPNYLLPKTQPELQWAWTNMEQYLSACLNLTERKRLVAQHLTHVSQTQ
EQNKENQNPNSDAPVIRSKTSARYMELVGWLVDKGITSEKQWIQEDQASY
ISFNAASNSRSQIKAALDNAGKIMSLTKTAPDYLVGQQPVEDISSNRIYK
ILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVR
VDQKCKSSAQIDPTPVIVTSNTNMCAVIDGNSTTFEHQQPLQDRMFKFEL
TRRLDHDFGKVTKQEVKDFFRWAKDHVVEVEHEFYVKKGGAKKRPAPSDA
DISEPKRVRESVAQPSTSDAEASINYADRLARGHSL (SEQ ID NO: 5
).

AAV2 Rep52 polypeptide sequence:

MELVGWLVDKGITSEKQWIQEDQASYISFNAASNSRSQIKAALDNAGKIM
SLTKTAPDYLVGQQPVEDISSNRIYKILELNGYDPQYAASVFLGWATKKF
GKRNTIWLFGPATTGKTNIAEAIAHTVPFYGCVNWTNENFPFNDCVDKMV
IWWEEGKMTAKVVESAKAILGGSKVRVDQKCKSSAQIDPTPVIVTSNTNM
CAVIDGNSTTFEHQQPLQDRMFKFELTRRLDHDFGKVTKQEVKDFFRWAK
DHVVEVEHEFYVKKGGAKKRPAPSDADISEPKRVRESVAQPSTSDAEASI
NYADRYQNKCSRHVGMNLMLFPCRQCERMNQNSNICFTHGQKDCLECFPV
SESQPVSVVKKAYQKLCYIHHIMGKVPDACTACDLVNVDLDDCIFEQ (S
EQ ID NO: 6).

AAV2 Rep40 polypeptide sequence:

MELVGWLVDKGITSEKQWIQEDQASYISFNAASNSRSQIKAALDNAGKIM
SLTKTAPDYLVGQQPVEDISSNRIYKILELNGYDPQYAASVFLGWATKKF
GKRNTIWLFGPATTGKTNIAEAIAHTVPFYGCVNWTNENFPFNDCVDKMV
IWWEEGKMTAKVVESAKAILGGSKVRVDQKCKSSAQIDPTPVIVTSNTNM
CAVIDGNSTTFEHQQPLQDRMFKFELTRRLDHDFGKVTKQEVKDFFRWAK
DHVVEVEHEFYVKKGGAKKRPAPSDADISEPKRVRESVAQPSTSDAEASI
NYADRLARGHSL (SEQ ID NO: 7).

In some embodiments, an AAV replication polypeptide is an AAV2 replication polypeptide. In various embodiments, the AAV replication polypeptide comprises or consists essentially of a polypeptide sequence having about or at least about 80%, 85%, 90%, 95%, 96%, 97%, 98%, 99%, or 100% identity to one of the AAV2 replication polypeptide sequences provided above. In various embodiments, the AAV replication polypeptide is AAV2 Rep78, AAV2 Rep68, AAV2 Rep52, AAV2 Rep40, or a combination thereof. Exemplary AAV2 Rep78, AAV2 Rep68, AAV2 Rep52, and AAV2 Rep40 polypeptide sequences are provided above. In various embodiments, the replication polypeptide is derived from a replication polypeptide from adeno-associated virus 2.

By “AAV Replication nucleic acid molecule (AAV Rep)” is meant a polynucleotide or a fragment thereof that encodes one or more AAV replication polypeptide(s). In various embodiments, the AAV Rep encodes each or a selection of the Rep78, Rep68, Rep52, and Rep40 polypeptide sequences listed above. In various embodiments, the AAV Rep comprises or consists essentially of a polynucleotide having about or at least about 80%, 85%, 90%, 95%, 96%, 97%, 98%, 99%, or 100% identity to the AAV2 Rep polynucleotide sequence provided below or to a fragment of the AAV2 Rep polynucleotide sequence provided below. In various embodiments, the fragment of the AAV2 Rep polynucleotide is about or at least about 800 bp, 900 bp, 1,000 bp, 1,100 bp, 1,200 bp, 1,300 bp, 1,400 bp, 1,500 bp, 1,600 bp, 1,700 bp, 1,800 bp, or 1,900 bp in length. In some embodiments, the AAV replication nucleic acid molecule is an AAV2 Rep. The following is an exemplary AAV2 Rep sequence:

ATGCCGGGGTTTTACGAGATTGTGATTAAGGTCCCCAGCGACCTTGACGA
GCATCTGCCCGGCATTTCTGACAGCTTTGTGAACTGGGTGGCCGAGAAGG
AATGGGAGTTGCCGCCAGATTCTGACATGGATCTGAATCTGATTGAGCAG
GCACCCCTGACCGTGGCCGAGAAGCTGCAGCGCGACTTTCTGACGGAATG
GCGCCGTGTGAGTAAGGCCCCGGAGGCTCTTTTCTTTGTGCAATTTGAGA
AGGGAGAGAGCTACTTCCACATGCACGTGCTCGTGGAAACCACCGGGGTG
AAATCCATGGTTTTGGGACGTTTCCTGAGTCAGATTCGCGAAAAACTGAT
TCAGAGAATTTACCGCGGGATCGAGCCGACTTTGCCAAACTGGTTCGCGG
TCACAAAGACCAGAAATGGCGCCGGAGGCGGGAACAAGGTGGTGGATGAG
TGCTACATCCCCAATTACTTGCTCCCCAAAACCCAGCCTGAGCTCCAGTG
GGCGTGGACTAATATGGAACAGTATTTAAGCGCCTGTTTGAATCTCACGG
AGCGTAAACGGTTGGTGGCGCAGCATCTGACGCACGTGTCGCAGACGCAG
GAGCAGAACAAAGAGAATCAGAATCCCAATTCTGATGCGCCGGTGATCAG
ATCAAAAACTTCAGCCAGGTACATGGAGCTGGTCGGGTGGCTCGTGGACA
AGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTT
GGACAATGCGGGAAAGATTATGAGCCTGACTAAAACCGCCCCCGACTACC
TGGTGGGCCAGCAGCCCGTGGAGGACATTTCCAGCAATCGGATTTATAAA
ATTTTGGAACTAAACGGGTACGATCCCCAATATGCGGCTTCCGTCTTTCT
GGGATGGGCCACGAAAAAGTTCGGCAAGAGGAACACCATCTGGCTGTTTG
GGCCTGCAACTACCGGGAAGACCAACATCGCGGAGGCCATAGCCCACACT
GTGCCCTTCTACGGGTGCGTAAACTGGACCAATGAGAACTTTCCCTTCAA
CGACTGTGTCGACAAGATGGTGATCTGGTGGGAGGAGGGGAAGATGACCG
CCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGAT
CGTCACCTCCAACACCAACATGTGCGCCGTGATTGACGGGAACTCAACGA
CCTTCGAACACCAGCAGCCGTTGCAAGACCGGATGTTCAAATTTGAACTC
ACCCGCCGTCTGGATCATGACTTTGGGAAGGTCACCAAGCAGGAAGTCAA
AGACTTTTTCCGGTGGGCAAAGGATCACGTGGTTGAGGTGGAGCATGAAT
TCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGAC
GTCAGACGCGGAAGCTTCGATCAACTACGCGGACAGGTACCAAAACAAAT
GTTCTCGTCACGTGGGCATGAATCTGATGCTGTTTCCCTGCAGACAATGC
GAGAGACTGAATCAGAATTCAAATATCTGCTTCACTCACGGTGTCAAAGA
CTGTTTAGAGTGCTTTCCCGTGTCAGAATCTCAACCCGTTTCTGTCGTCA
AAAAGGCGTATCAGAAACTGTGCTACATTCATCACATCATGGGAAAGGTG
CCAGACGCTTGCACTGCTTGCGACCTGGTCAATGTGGACTTGGATGACTG
TGTTTCTGAACAATAA (SEQ ID NO: 8).

By “AAV-Retro capsid polypeptide” is meant an adeno-associated virus capsid protein that alone or together with other proteins functions to facilitate or improve retrograde infection (Retro) of a cell by a virus particle comprising the polypeptide. In some embodiments, the AAV-Retro capsid polypeptide comprises the amino acid sequence LAxxDxTKxA (SEQ ID NO: 1) or LAxDxTKxxA (SEQ ID NO: 2), where X is any amino acid or is absent. In some embodiments, the AAV-Retro capsid polypeptide comprises or consists essentially of one of the polypeptide sequences provided below. In some embodiments, an AAV-Retro capsid polypeptide comprises or consists essentially of an AAV7, AAV9, or AAV10 replication polypeptide. In various embodiments, the AAV replication polypeptide comprises or consists essentially of a polypeptide sequence having about or at least about 80%, 85%, 90%, 95%, 96%, 97%, 98%, 99%, or 100% identity to one of the AAV9Retro VP1, AAV9Retro VP2, AAV9Retro VP3, AAV9 VP1, AAV9 VP2, AAV9 VP3, AAV10Retro VP1, AAV10Retro VP2, AAV10 VP3, AAV10 VP1, AAV10 VP2, and AAV10 VP3 polypeptide sequences provided below. In various embodiments, the AAV capsid polypeptide is AAV9Retro VP1, AAV9Retro VP2, AAV9Retro VP3, AAV9 VP1, AAV9 VP2, AAV9 VP3, AAV10Retro VP1, AAV10Retro VP2, AAV10 VP3, AAV10 VP1, AAV10 VP2, AAV10 VP3, or a combination thereof. Exemplary AAV9Retro VP1, AAV9Retro VP2, AAV9Retro VP3, AAV9 VP1, AAV9 VP2, AAV9 VP3, AAV10Retro VP1, AAV10Retro VP2, AAV10 VP3, AAV10 VP1, AAV10 VP2, and AAV10 VP3 polypeptide sequences are provided below.

AAV9Retro VP1 polypeptide sequence:

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQLADQDYTKTALA
DQDYTKTAAQAQTGWVQNQGILPGMVWQDRDVYLQGPIWAKIPHTDGNFH
PSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTR
YLTRNL(SEQ ID NO: 9).

AAV9 VP1 polypeptide sequence:

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQLADQDYTKTAAQ
AQTGWVQNQGILPGMVWQDRDVYLQGPIWAKIPHTDGNFHPSPLMGGFGM
KHPPPQILIKNTPVPADPPTAFNKDKLNSFITQYSTGQVSVEIEWELQKE
NSKRWNPEIQYTSNYYKSNNVEFAVNTEGVYSEPRPIGTRYLTRNL (SE
Q ID NO:10).

AAV9Retro VP2 polypeptide sequence:

TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSA
QLADQDYTKTAAQAQTGWVQNQGILPGMVWQDRDVYLQGPIWAKIPHTDG
NFHPSPLMGGFGMKHPPPQILIKNTPVPADPPTAFNKDKLNSFITQYSTG
QVSVEIEWELQKENSKRWNPEIQYTSNYYKSNNVEFAVNTEGVYSEPRPI
GTRYLTRNL (SEQ ID NO: 11).

AAV9 VP2 polypeptide sequence:

TAPGKKRPVEQSPQEPDSSAGIGKSGAQPAKKRLNFGQTGDTESVPDPQP
IGEPPAAPSGVGSLTMASGGGAPVADNNEGADGVGSSSGNWHCDSQWLGD
RVITTSTRTWALPTYNNHLYKQISNSTSGGSSNDNAYFGYSTPWGYFDFN
RFHCHFSPRDWQRLINNNWGFRPKRLNFKLFNIQVKEVTDNNGVKTIANN
LTSTVQVFTDSDYQLPYVLGSAHEGCLPPFPADVFMIPQYGYLTLNDGSQ
AVGRSSFYCLEYFPSQMLRTGNNFQFSYEFENVPFHSSYAHSQSLDRLMN
PLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPL
SGSLIFGKQGTGRDNVDADKVMITNEEEIKTTNPVATESYGQVATNHQSA
QAQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWEL
QKENSKRWNPEIQYTSNYYKSNNVEFAVNTEGVYSEPRPIGTRYLTRNL
(SEQ ID NO: 12).

AAV9Retro VP3 polypeptide sequence:

MASGGGAPVADNNEGADGVGSSSGNWHCDSQWLGDRVITTSTRTWALPTY
NNHLYKQISNSTSGGSSNDNAYFGYSTPWGYFDFNRFHCHFSPRDWQRLI
NNNWGFRPKRLNFKLFNIQVKEVTDNNGVKTIANNLTSTVQVFTDSDYQL
PYVLGSAHEGCLPPFPADVFMIPQYGYLTLNDGSQAVGRSSFYCLEYFPS
QMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFA
WPGASSWALNGRNSLMNPGPAMASHKEGEDRFFPLSGSLIFGKQGTGRDN
VDADKVMITNEEEIKTTNPVATESYGQVATNHQSAQLADQDYTKTAAQAQ
TGWVQNQGILPGMVWQDRDVYLQGPIWAKIPHTDGNFHPSPLMGGFGMKH
PPPQILIKNTPVPADPPTAFNKDKLNSFITQYSTGQVSVEIEWELQKENS
KRWNPEIQYTSNYYKSNNVEFAVNTEGVYSEPRPIGTRYLTRNL (SEQ
ID NO: 13).

AAV9 VP3 polypeptide sequence:

MASGGGAPVADNNEGADGVGSSSGNWHCDSQWLGDRVITTSTRTWALPTY
NNHLYKQISNSTSGGSSNDNAYFGYSTPWGYFDFNRFHCHFSPRDWQRLI
NNNWGFRPKRLNFKLFNIQVKEVTDNNGVKTIANNLTSTVQVFTDSDYQL
PYVLGSAHEGCLPPFPADVFMIPQYGYLTLNDGSQAVGRSSFYCLEYFPS
QMLRTGNNFQFSYEFENVPFHSSYAHSQSLDRLMNPLIDQYLYYLSKTIN
GSGQNQQTLKFSVAGPSNMAVQGRNYIPGPSYRQQRVSTTVTQNNNSEFA
WPGASSWALNGRNSLMNPGPAMASHKEGEDRFFPLSGSLIFGKQGTGRDN
VDADKVMITNEEEIKTTNPVATESYGQVATNHQSAQAQAQTGWVQNQGIL
PGMVWQDRDVYLQGPIWAKIPHTDGNFHPSPLMGGFGMKHPPPQILIKNT
PVPADPPTAFNKDKLNSFITQYSTGQVSVEIEWELQKENSKRWNPEIQYT
SNYYKSNNVEFAVNTEGVYSEPRPIGTRYLTRNL (SEQ ID NO: 14)
.

AAV10Retro VP1 polypeptide sequence:

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNLADQDYTKTA
AAPIVGAVNSQGALPGMVWQNRDVYLQGPIWAKIPHTDGNFHPSPLMGGF
GLKHPPPQILIKNTPVPADPPTTFSQAKLASFITQYSTGQVSVEIEWELQ
KENSKRWNPEIQYTSNYYKSTNVDFAVNTDGTYSEPRPIGTRYLTRNL (
SEQ ID NO:15).

AAV10 VP1 polypeptide sequence:

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFSQAKLASFITQYSTGQVSVEIEWELQKENSKRWNPE
IQYTSNYYKSTNVDFAVNTDGTYSEPRPIGTRYLTRNL (SEQ ID NO:
16).

AAV10Retro VP2 polypeptide sequence:

TGIGKKGQQPAKKRLNFGQTGDSESVPDPQPIGEPPAGPSGLGSGTMAAG
GGAPMADNNEGADGVGSSSGNWHCDSTWLGDRVITTSTRTWALPTYNNHL
YKQISNGTSGGSTNDNTYFGYSTPWGYFDFNRFHCHFSPRDWQRLINNNW
GFRPKRLNFKLFNIQVKEVTQNEGTKTIANNLTSTIQVFTDSEYQLPYVL
GSAHQGCLPPFPADVFMIPQYGYLTLNNGSQAVGRSSFYCLEYFPSQMLR
TGNNFEFSYQFEDVPFHSSYAHSQSLDRLMNPLIDQYLYYLSRTQSTGGT
AGTQQLLFSQAGPNNMSAQAKNWLPGPCYRQQRVSTTLSQNNNSNFAWTG
ATKYHLNGRDSLVNPGVAMATHKDDEERFFPSSGVLMFGKQGAGKDNVDY
SSVMLTSEEEIKTTNPVATEQYGVVADNLQQQNLADQDYTKTAAAPIVGA
VNSQGALPGMVWQNRDVYLQGPIWAKIPHTDGNFHPSPLMGGFGLKHPPP
QILIKNTPVPADPPTTFSQAKLASFITQYSTGQVSVEIEWELQKENSKRW
NPEIQYTSNYYKSTNVDFAVNTDGTYSEPRPIGTRYLTRNL (SEQ ID
NO: 17).

AAV10 VP2 polypeptide sequence:

TGIGKKGQQPAKKRLNFGQTGDSESVPDPQPIGEPPAGPSGLGSGTMAAG
GGAPMADNNEGADGVGSSSGNWHCDSTWLGDRVITTSTRTWALPTYNNHL
YKQISNGTSGGSTNDNTYFGYSTPWGYFDFNRFHCHFSPRDWQRLINNNW
GFRPKRLNFKLFNIQVKEVTQNEGTKTIANNLTSTIQVFTDSEYQLPYVL
GSAHQGCLPPFPADVFMIPQYGYLTLNNGSQAVGRSSFYCLEYFPSQMLR
TGNNFEFSYQFEDVPFHSSYAHSQSLDRLMNPLIDQYLYYLSRTQSTGGT
AGTQQLLFSQAGPNNMSAQAKNWLPGPCYRQQRVSTTLSQNNNSNFAWTG
ATKYHLNGRDSLVNPGVAMATHKDDEERFFPSSGVLMFGKQGAGKDNVDY
SSVMLTSEEEIKTTNPVATEQYGVVADNLQQQNAAPIVGAVNSQGALPGM
VWQNRDVYLQGPIWAKIPHTDGNFHPSPLMGGFGLKHPPPQILIKNTPVP
ADPPTTFSQAKLASFITQYSTGQVSVEIEWELQKENSKRWNPEIQYTSNY
YKSTNVDFAVNTDGTYSEPRPIGTRYLTRNL (SEQID NO: 18).

AAV10Retro VP3 polypeptide sequence:

MAAGGGAPMADNNEGADGVGSSSGNWHCDSTWLGDRVITTSTRTWALPTY
NNHLYKQISNGTSGGSTNDNTYFGYSTPWGYFDFNRFHCHFSPRDWQRLI
NNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPS
QMLRTGNNFEFSYQFEDVPFHSSYAHSQSLDRLMNPLIDQYLYYLSRTQS
TGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKD
NVDYSSVMLTSEEEIKTTNPVATEQYGVVADNLQQQNLADQDYTKTAAAP
IVGAVNSQGALPGMVWQNRDVYLQGPIWAKIPHTDGNFHPSPLMGGFGLK
HPPPQILIKNTPVPADPPTTFSQAKLASFITQYSTGQVSVEIEWELQKEN
SKRWNPEIQYTSNYYKSTNVDFAVNTDGTYSEPRPIGTRYLTRNL (SEQ
ID NO: 19).

AAV10 VP3 polypeptide sequence:

MAAGGGAPMADNNEGADGVGSSSGNWHCDSTWLGDRVITTSTRTWALPTY
NNHLYKQISNGTSGGSTNDNTYFGYSTPWGYFDFNRFHCHFSPRDWQRLI
NNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPS
QMLRTGNNFEFSYQFEDVPFHSSYAHSQSLDRLMNPLIDQYLYYLSRTQS
TGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKD
NVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKN
TPVPADPPTTFSQAKLASFITQYSTGQVSVEIEWELQKENSKRWNPEIQY
TSNYYKSTNVDFAVNTDGTYSEPRPIGTRYLTRNL (SEQ ID NO: 20
).

By “AAV-Retro Capsid polynucleotide (AAV-Retro Cap)” is meant a polynucleotide or a fragment thereof that encodes one or more polypeptides that form part of a viral capsid. In various embodiments, the capsid encapsulates a viral polynucleotide (e.g., viral genome, which may comprise RNA and/or DNA). In some embodiments, the capsid encapsulates a heterologous polynucleotide sequence. In some embodiments, an AAV-Retro Cap is an AAV9Retro Cap or an AAV10Retro Cap. In various embodiments, the AAV Rep comprises or consists essentially of a polynucleotide sequence encoding a polypeptide sequence having about or at least about 80%, 85%, 90%, 95%, 96%, 97%, 98%, 99%, or 100% identity to one of the AAV9Retro VP1, AAV9Retro VP2, AAV9Retro VP3, AAV9 VP1, AAV9 VP2, AAV9 VP3, AAV10Retro VP1, AAV10Retro VP2, AAV10 VP3, AAV10 VP1, AAV10 VP2, and AAV10 VP3 polypeptide sequences provided above.

In various embodiments, the AAV-Retro Cap encodes a capsid derived from a virus of the same serotype as adeno-associated virus serotype 9 (i.e., is an AAV9Retro Cap). The sequence of an exemplary AAV9Retro Cap polynucleotide sequence is provided below. In various embodiments, the AAV9Retro Cap polynucleotide sequence comprises a nucleotide sequence with about or at least about 80%, 85%, 90%, 95%, 96%, 97%, 98%, 99%, or 100% identity to the exemplary AAV9Retro Cap polynucleotide sequence provided below. In various embodiments, the AAV9Retro VP1, AAV9Retro VP2, AAV9Retro VP3, AAV9 VP1, AAV9 VP2, and/or AAV9 VP3 polypeptide is encoded by a polynucleotide sequence with about or at least about 80%, 85%, 90%, 95%, 96%, 97%, 98%, 99%, or 100% identity to the AAV9Retro Cap polynucleotide sequence provided below, or a fragment thereof. In various embodiments, the AAV9Retro VP1, AAV9Retro VP2, AAV9Retro VP3, AAV9 VP1, AAV9 VP2, AAV9 VP3, AAV10Retro VP1, AAV10Retro VP2, AAV10 VP3, AAV10 VP1, AAV10 VP2, and/or AAV10 VP3 polypeptide is encoded by a polynucleotide sequence with about or at least about 80%, 85%, 90%, 95%, 96%, 97%, 98%, 99%, or 100% identity to the AAV9 Cap polynucleotide sequence provided below, or a fragment thereof. In various embodiments, the fragment of the AAV9Retro Cap polynucleotide sequence or the AAV9 CAP polynucleotide sequence is about or at least about 1,000 bp, 1,100 bp, 1,200 bp, 1,300 bp, 1,400 bp, 1,500 bp, 1,600 bp, 1,700 bp, 1,800 bp, 1,900 bp, 2,000 bp, 2,100 bp, 2,200 bp, 2,300 bp, 2,400 bp, or 2,500 bp in length. In various embodiments, the AAV9Retro Cappolynucleotide comprises a sequence with about or at least about 80%, 85%, 90%, 95%, 96%, 97%, 98%, 99%, or 100% identity to the nucleotide sequence CTAGCAGACCAAGACTACACAAAAACTGCT (SEQ ID NO: 21). In various embodiments, the AAV9Retro Cap encodes a virion protein 1 (VP1) polypeptide sequence comprising an amino acid sequence with about or at least about 80%, 85%, 90%, 95%, 96%, 97%, 98%, 99%, or 100% identity to the amino acid sequence LADQDYTKTA (SEQ ID NO: 3).

AAV9Retro Cap polynucleotide sequence:

ATGGCTGCCGATGGTTATCTTCCAGATTGGCTCGAGGACAACCTTAGTGA
AGGAATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCAAAGACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGAACTGGAAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCCCAACTAGCAGACCAAGACTACACAAAAACTGCTGCACAG
GCGCAGACCGGCTGGGTTCAAAACCAAGGAATACTTCCGGGTATGGTTTG
GCAGGACAGAGATGTGTACCTGCAAGGACCCATTTGGGCCAAAATTCCTC
ACACGGACGGCAACTTTCACCCTTCTCCGCTGATGGGAGGGTTTGGAATG
AAGCACCCGCCTCCTCAGATCCTCATCAAAAACACACCTGTACCTGCGGA
TCCTCCAACGGCCTTCAACAAGGACAAGCTGAACTCTTTCATCACCCAGT
ATTCTACTGGCCAAGTCAGCGTGGAGATCGAGTGGGAGCTGCAGAAGGAA
AACAGCAAGCGCTGGAACCCGGAGATCCAGTACACTTCCAACTATTACAA
GTCTAATAATGTTGAATTTGCTGTTAATACTGAAGGTGTATATAGTGAAC
CCCGCCCCATTGGCACCAGATACCTGACTCGTAATCTGTAA (SEQ ID
NO: 22).

AAV9 Cap polynucleotide sequence:

ATGGCTGCCGATGGTTATCTTCCAGATTGGCTCGAGGACAACCTTAGTGA
AGGAATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCAAAGACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGAACTGGAAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACC
CATTTGGGCCAAAATTCCTCACACGGACGGCAACTTTCACCCTTCTCCGC
TGATGGGAGGGTTTGGAATGAAGCACCCGCCTCCTCAGATCCTCATCAAA
AACACACCTGTACCTGCGGATCCTCCAACGGCCTTCAACAAGGACAAGCT
GAACTCTTTCATCACCCAGTATTCTACTGGCCAAGTCAGCGTGGAGATCG
AGTGGGAGCTGCAGAAGGAAAACAGCAAGCGCTGGAACCCGGAGATCCAG
TACACTTCCAACTATTACAAGTCTAATAATGTTGAATTTGCTGTTAATAC
TGAAGGTGTATATAGTGAACCCCGCCCCATTGGCACCAGATACCTGACTC
GTAATCTGTAA (SEQ ID NO: 23).

In various embodiments, the AAV-Retro Cap encodes a capsid derived from a virus of the same serotype as adeno-associated virus serotype 10 (i.e., is an AAV10Retro Cap). The sequence of an exemplary AAV10Retro Cap polynucleotide sequence is provided below. In various embodiments, the AAV10Retro Cap polynucleotide sequence comprises a nucleotide sequence with about or at least about 80%, 85%, 90%, 95%, 96%, 97%, 98%, 99%, or 100% identity to the exemplary AAV10Retro Cap polynucleotide sequence provided below.. In various embodiments, the AAV10Retro VP1, AAV10Retro VP2, AAV10Retro VP3, AAV10 VP1, AAV10 VP2, and/or AAV10 VP3 polypeptide is encoded by a polynucleotide sequence with about or at least about 80%, 85%, 90%, 95%, 96%, 97%, 98%, 99%, or 100% identity to the AAV10Retro Cap polynucleotide sequence provided below, or a fragment thereof. In various embodiments, the AAV10Retro VP1, AAV10Retro VP2, AAV10Retro VP3, AAV10 VP1, AAV10 VP2, and/or AAV10 VP3 polypeptide is encoded by a polynucleotide sequence with about or at least about 80%, 85%, 90%, 95%, 96%, 97%, 98%, 99%, or 100% identity to the AAV10 Cap polynucleotide sequence provided below, or a fragment thereof. In various embodiments, the fragment of the AAV10Retro Cap or the AAV10 CAP is about or at least about 1,000 bp, 1,100 bp, 1,200 bp, 1,300 bp, 1,400 bp, 1,500 bp, 1,600 bp, 1,700 bp, 1,800 bp, 1,900 bp, 2,000 bp, 2,100 bp, 2,200 bp, 2,300 bp, 2,400 bp, or 2,500 bp in length. In various embodiments, the AAV10Retro Cap polynucleotide comprises a sequence with about or at least about 80%, 85%, 90%, 95%, 96%, 97%, 98%, 99%, or 100% identity to the nucleotide sequence CTAGCAGACCAAGACTACACAAAAACTGCT (SEQ ID NO: 21). In various embodiments, the AAV10Retro Cap encodes a virion protein 1 (VP1) polypeptide sequence comprising an amino acid sequence with about or at least about 80%, 85%, 90%, 95%, 96%, 97%, 98%, 99%, or 100% identity to the amino acid sequence LADQDYTKTA (SEQ ID NO: 3).

AAV10Retro Cap polynucleotide sequence:

ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCCATCACCC
CAGCGTTCTCCAGACTCCTCTACGGGCATCGGCAAGAAAGGCCAGCAGCC
CGCGAAAAAGAGACTCAACTTTGGGCAGACTGGCGACTCAGAGTCAGTGC
CCGACCCTCAACCAATCGGAGAACCCCCCGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGAACCTGGGCCCTC
CCCACCTACAACAACCACCTCTACAAGCAAATCTCCAACGGGACTTCGGG
AGGAAGCACCAACGACAACACCTACTTCGGCTACAGCACCCCCTGGGGGT
ATTTTGACTTTAACAGATTCCACTGCCACTTCTCACCACGTGACTGGCAG
CGACTCATCAACAACAACTGGGGATTCCGGCCCAAGAGACTCAACTTCAA
GCTCTTCAACATCCAGGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTCCTCGGCTCTGCGCACCAGGGCTGCCTGCCTCC
GTTCCCGGCGGACGTCTTCATGATTCCTCAGTACGGGTACCTGACTCTGA
ACAATGGCAGTCAGGCCGTGGGCCGTTCCTCCTTCTACTGCCTGGAGTAC
TTTCCTTCTCAAATGCTGAGAACGGGCAACAACTTTGAGTTCAGCTACCA
GTTTGAGGACGTGCCTTTTCACAGCAGCTACGCGCACAGCCAAAGCCTGG
ACCGGCTGATGAACCCCCTCATCGACCAGTACCTGTACTACCTGTCTCGG
ACTCAGTCCACGGGAGGTACCGCAGGAACTCAGCAGTTGCTATTTTCTCA
GGCCGGGCCTAATAACATGTCGGCTCAGGCCAAAAACTGGCTACCCGGGC
CCTGCTACCGGCAGCAACGCGTCTCCACGACACTGTCGCAAAATAACAAC
AGCAACTTTGCCTGGACCGGTGCCACCAAGTATCATCTGAATGGCAGAGA
CTCTCTGGTAAATCCCGGTGTCGCTATGGCAACCCACAAGGACGACGAAG
AGCGATTTTTTCCGTCCAGCGGAGTCTTAATGTTTGGGAAACAGGGAGCT
GGAAAAGACAACGTGGACTATAGCAGCGTTATGCTAACCAGTGAGGAAGA
AATTAAAACCACCAACCCAGTGGCCACAGAACAGTACGGCGTGGTGGCCG
ATAACCTGCAACAGCAAAACCTAGCAGACCAAGACTACACAAAAACTGCT
GCCGCTCCTATTGTAGGGGCCGTCAACAGTCAAGGAGCCTTACCTGGCAT
GGTCTGGCAGAACCGGGACGTGTACCTGCAGGGTCCTATCTGGGCCAAGA
TTCCTCACACGGACGGAAACTTTCATCCCTCGCCGCTGATGGGAGGCTTT
GGACTGAAACACCCGCCTCCTCAGATCCTGATTAAGAATACACCTGTTCC
CGCGGATCCTCCAACTACCTTCAGTCAAGCTAAGCTGGCGTCGTTCATCA
CGCAGTACAGCACCGGACAGGTCAGCGTGGAAATTGAATGGGAGCTGCAG
AAAGAAAACAGCAAACGCTGGAACCCAGAGATTCAATACACTTCCAACTA
CTACAAATCTACAAATGTGGACTTTGCTGTTAACACAGATGGCACTTATT
(SEQ ID NO: 24).

AAV10 Cap polynucleotide sequence:

ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCCATCACCC
CAGCGTTCTCCAGACTCCTCTACGGGCATCGGCAAGAAAGGCCAGCAGCC
CGCGAAAAAGAGACTCAACTTTGGGCAGACTGGCGACTCAGAGTCAGTGC
CCGACCCTCAACCAATCGGAGAACCCCCCGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGAACCTGGGCCCTC
CCCACCTACAACAACCACCTCTACAAGCAAATCTCCAACGGGACTTCGGG
AGGAAGCACCAACGACAACACCTACTTCGGCTACAGCACCCCCTGGGGGT
ATTTTGACTTTAACAGATTCCACTGCCACTTCTCACCACGTGACTGGCAG
CGACTCATCAACAACAACTGGGGATTCCGGCCCAAGAGACTCAACTTCAA
GCTCTTCAACATCCAGGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTCCTCGGCTCTGCGCACCAGGGCTGCCTGCCTCC
GTTCCCGGCGGACGTCTTCATGATTCCTCAGTACGGGTACCTGACTCTGA
ACAATGGCAGTCAGGCCGTGGGCCGTTCCTCCTTCTACTGCCTGGAGTAC
TTTCCTTCTCAAATGCTGAGAACGGGCAACAACTTTGAGTTCAGCTACCA
GTTTGAGGACGTGCCTTTTCACAGCAGCTACGCGCACAGCCAAAGCCTGG
ACCGGCTGATGAACCCCCTCATCGACCAGTACCTGTACTACCTGTCTCGG
ACTCAGTCCACGGGAGGTACCGCAGGAACTCAGCAGTTGCTATTTTCTCA
GGCCGGGCCTAATAACATGTCGGCTCAGGCCAAAAACTGGCTACCCGGGC
CCTGCTACCGGCAGCAACGCGTCTCCACGACACTGTCGCAAAATAACAAC
AGCAACTTTGCCTGGACCGGTGCCACCAAGTATCATCTGAATGGCAGAGA
CTCTCTGGTAAATCCCGGTGTCGCTATGGCAACCCACAAGGACGACGAAG
AGCGATTTTTTCCGTCCAGCGGAGTCTTAATGTTTGGGAAACAGGGAGCT
GGAAAAGACAACGTGGACTATAGCAGCGTTATGCTAACCAGTGAGGAAGA
AATTAAAACCACCAACCCAGTGGCCACAGAACAGTACGGCGTGGTGGCCG
ATAACCTGCAACAGCAAAACGCCGCTCCTATTGTAGGGGCCGTCAACAGT
CAAGGAGCCTTACCTGGCATGGTCTGGCAGAACCGGGACGTGTACCTGCA
GGGTCCTATCTGGGCCAAGATTCCTCACACGGACGGAAACTTTCATCCCT
CGCCGCTGATGGGAGGCTTTGGACTGAAACACCCGCCTCCTCAGATCCTG
ATTAAGAATACACCTGTTCCCGCGGATCCTCCAACTACCTTCAGTCAAGC
TAAGCTGGCGTCGTTCATCACGCAGTACAGCACCGGACAGGTCAGCGTGG
AAATTGAATGGGAGCTGCAGAAAGAAAACAGCAAACGCTGGAACCCAGAG
ATTCAATACACTTCCAACTACTACAAATCTACAAATGTGGACTTTGCTGT
TAACACAGATGGCACTTATT (SEQ ID NO: 25).

By “AAV2/9Retro vector” is meant a viral vector comprising an AAV9Retro capsid polypeptide or fragment thereof and that transfects a cell of the central nervous system. In some embodiments, the cell is a nerve cell, an astrocyte, a pericyte, a microglial cell, an oligodendrocyte, or a combination thereof. In some embodiments, the nerve cell is a projection neuron. In various embodiments, the AAV2/9Retro vector targets a projection neuron for retrograde infection. In some embodiments, the AAV2/9Retro vector selectively targets damaged neurons where the damage in some embodiments is caused by a traumatic insult. In some embodiments, the AAV2/9Retro vector crosses the blood brain barrier (BBB) or the blood spinal cord barrier (BSCB) at site where neurons are damaged and/or where the BBB or BSCB is damaged, optionally where the damage is caused by a traumatic insult. The sequence of an exemplary plasmid encoding an AAV2/9Retro vector is provided below. In various embodiments, the plasmid encoding the AAV2/9Retro vector comprises a nucleotide sequence with about or at least about 80%, 85%, 90%, 95%, 96%, 97%, 98%, 99%, or 100% identity to the exemplary AAV2/9Retro vector-encoding plasmid sequence provided below. In various embodiments, the AAV2/9Retro vector encodes an AAV2/9Retro virus.

AAV2/9Retro vector-encoding plasmid sequence:

GTCGACGGTATCGGGGGAGCTCGCAGGGTCTCCATTTTGAAGCGGGAGGT
TTGAACGCGCAGCCGCCATGCCGGGGTTTTACGAGATTGTGATTAAGGTC
CCCAGCGACCTTGACGAGCATCTGCCCGGCATTTCTGACAGCTTTGTGAA
CTGGGTGGCCGAGAAGGAATGGGAGTTGCCGCCAGATTCTGACATGGATC
TGAATCTGATTGAGCAGGCACCCCTGACCGTGGCCGAGAAGCTGCAGCGC
GACTTTCTGACGGAATGGCGCCGTGTGAGTAAGGCCCCGGAGGCTCTTTT
CTTTGTGCAATTTGAGAAGGGAGAGAGCTACTTCCACATGCACGTGCTCG
TGGAAACCACCGGGGTGAAATCCATGGTTTTGGGACGTTTCCTGAGTCAG
ATTCGCGAAAAACTGATTCAGAGAATTTACCGCGGGATCGAGCCGACTTT
GCCAAACTGGTTCGCGGTCACAAAGACCAGAAATGGCGCCGGAGGCGGGA
ACAAGGTGGTGGATGAGTGCTACATCCCCAATTACTTGCTCCCCAAAACC
CAGCCTGAGCTCCAGTGGGCGTGGACTAATATGGAACAGTATTTAAGCGC
CTGTTTGAATCTCACGGAGCGTAAACGGTTGGTGGCGCAGCATCTGACGC
ACGTGTCGCAGACGCAGGAGCAGAACAAAGAGAATCAGAATCCCAATTCT
GATGCGCCGGTGATCAGATCAAAAACTTCAGCCAGGTACATGGAGCTGGT
CGGGTGGCTCGTGGACAAGGGGATTACCTCGGAGAAGCAGTGGATCCAGG
AGGACCAGGCCTCATACATCTCCTTCAATGCGGCCTCCAACTCGCGGTCC
CAAATCAAGGCTGCCTTGGACAATGCGGGAAAGATTATGAGCCTGACTAA
AACCGCCCCCGACTACCTGGTGGGCCAGCAGCCCGTGGAGGACATTTCCA
GCAATCGGATTTATAAAATTTTGGAACTAAACGGGTACGATCCCCAATAT
GCGGCTTCCGTCTTTCTGGGATGGGCCACGAAAAAGTTCGGCAAGAGGAA
CACCATCTGGCTGTTTGGGCCTGCAACTACCGGGAAGACCAACATCGCGG
AGGCCATAGCCCACACTGTGCCCTTCTACGGGTGCGTAAACTGGACCAAT
GAGAACTTTCCCTTCAACGACTGTGTCGACAAGATGGTGATCTGGTGGGA
GGAGGGGAAGATGACCGCCAAGGTCGTGGAGTCGGCCAAAGCCATTCTCG
GAGGAAGCAAGGTGCGCGTGGACCAGAAATGCAAGTCCTCGGCCCAGATA
GACCCGACTCCCGTGATCGTCACCTCCAACACCAACATGTGCGCCGTGAT
TGACGGGAACTCAACGACCTTCGAACACCAGCAGCCGTTGCAAGACCGGA
TGTTCAAATTTGAACTCACCCGCCGTCTGGATCATGACTTTGGGAAGGTC
ACCAAGCAGGAAGTCAAAGACTTTTTCCGGTGGGCAAAGGATCACGTGGT
TGAGGTGGAGCATGAATTCTACGTCAAAAAGGGTGGAGCCAAGAAAAGAC
CCGCCCCCAGTGACGCAGATATAAGTGAGCCCAAACGGGTGCGCGAGTCA
GTTGCGCAGCCATCGACGTCAGACGCGGAAGCTTCGATCAACTACGCGGA
CAGGTACCAAAACAAATGTTCTCGTCACGTGGGCATGAATCTGATGCTGT
TTCCCTGCAGACAATGCGAGAGACTGAATCAGAATTCAAATATCTGCTTC
ACTCACGGTGTCAAAGACTGTTTAGAGTGCTTTCCCGTGTCAGAATCTCA
ACCCGTTTCTGTCGTCAAAAAGGCGTATCAGAAACTGTGCTACATTCATC
ACATCATGGGAAAGGTGCCAGACGCTTGCACTGCTTGCGACCTGGTCAAT
GTGGACTTGGATGACTGTGTTTCTGAACAATAAATGACTTAAACCAGGTA
TGGCTGCCGATGGTTATCTTCCAGATTGGCTCGAGGACAACCTTAGTGAA
GGAATTCGCGAGTGGTGGGCTTTGAAACCTGGAGCCCCTCAACCCAAGGC
AAATCAACAACATCAAGACAACGCTCGAGGTCTTGTGCTTCCGGGTTACA
AATACCTTGGACCCGGCAACGGACTCGACAAGGGGGAGCCGGTCAACGCA
GCAGACGCGGCGGCCCTCGAGCACGACAAGGCCTACGACCAGCAGCTCAA
GGCCGGAGACAACCCGTACCTCAAGTACAACCACGCCGACGCCGAGTTCC
AGGAGCGGCTCAAAGAAGATACGTCTTTTGGGGGCAACCTCGGGCGAGCA
GTCTTCCAGGCCAAAAAGAGGCTTCTTGAACCTCTTGGTCTGGTTGAGGA
AGCGGCTAAGACGGCTCCTGGAAAGAAGAGGCCTGTAGAGCAGTCTCCTC
AGGAACCGGACTCCTCCGCGGGTATTGGCAAATCGGGTGCACAGCCCGCT
AAAAAGAGACTCAATTTCGGTCAGACTGGCGACACAGAGTCAGTCCCAGA
CCCTCAACCAATCGGAGAACCTCCCGCAGCCCCCTCAGGTGTGGGATCTC
TTACAATGGCTTCAGGTGGTGGCGCACCAGTGGCAGACAATAACGAAGGT
GCCGATGGAGTGGGTAGTTCCTCGGGAAATTGGCATTGCGATTCCCAATG
GCTGGGGGACAGAGTCATCACCACCAGCACCCGAACCTGGGCCCTGCCCA
CCTACAACAATCACCTCTACAAGCAAATCTCCAACAGCACATCTGGAGGA
TCTTCAAATGACAACGCCTACTTCGGCTACAGCACCCCCTGGGGGTATTT
TGACTTCAACAGATTCCACTGCCACTTCTCACCACGTGACTGGCAGCGAC
TCATCAACAACAACTGGGGATTCCGGCCTAAGCGACTCAACTTCAAGCTC
TTCAACATTCAGGTCAAAGAGGTTACGGACAACAATGGAGTCAAGACCAT
CGCCAATAACCTTACCAGCACGGTCCAGGTCTTCACGGACTCAGACTATC
AGCTCCCGTACGTGCTCGGGTCGGCTCACGAGGGCTGCCTCCCGCCGTTC
CCAGCGGACGTTTTCATGATTCCTCAGTACGGGTATCTGACGCTTAATGA
TGGAAGCCAGGCCGTGGGTCGTTCGTCCTTTTACTGCCTGGAATATTTCC
CGTCGCAAATGCTAAGAACGGGTAACAACTTCCAGTTCAGCTACGAGTTT
GAGAACGTACCTTTCCATAGCAGCTACGCTCACAGCCAAAGCCTGGACCG
ACTAATGAATCCACTCATCGACCAATACTTGTACTATCTCTCAAAGACTA
TTAACGGTTCTGGACAGAATCAACAAACGCTAAAATTCAGTGTGGCCGGA
CCCAGCAACATGGCTGTCCAGGGAAGAAACTACATACCTGGACCCAGCTA
CCGACAACAACGTGTCTCAACCACTGTGACTCAAAACAACAACAGCGAAT
TTGCTTGGCCTGGAGCTTCTTCTTGGGCTCTCAATGGACGTAATAGCTTG
ATGAATCCTGGACCTGCTATGGCCAGCCACAAAGAAGGAGAGGACCGTTT
CTTTCCTTTGTCTGGATCTTTAATTTTTGGCAAACAAGGAACTGGAAGAG
ACAACGTGGATGCGGACAAAGTCATGATAACCAACGAAGAAGAAATTAAA
ACTACTAACCCGGTAGCAACGGAGTCCTATGGACAAGTGGCCACAAACCA
CCAGAGTGCCCAACTAGCAGACCAAGACTACACAAAAACTGCTGCACAGG
CGCAGACCGGCTGGGTTCAAAACCAAGGAATACTTCCGGGTATGGTTTGG
CAGGACAGAGATGTGTACCTGCAAGGACCCATTTGGGCCAAAATTCCTCA
CACGGACGGCAACTTTCACCCTTCTCCGCTGATGGGAGGGTTTGGAATGA
AGCACCCGCCTCCTCAGATCCTCATCAAAAACACACCTGTACCTGCGGAT
CCTCCAACGGCCTTCAACAAGGACAAGCTGAACTCTTTCATCACCCAGTA
TTCTACTGGCCAAGTCAGCGTGGAGATCGAGTGGGAGCTGCAGAAGGAAA
ACAGCAAGCGCTGGAACCCGGAGATCCAGTACACTTCCAACTATTACAAG
TCTAATAATGTTGAATTTGCTGTTAATACTGAAGGTGTATATAGTGAACC
CCGCCCCATTGGCACCAGATACCTGACTCGTAATCTGTAATTGCTTGTTA
ATCAATAAACCGTTTAATTCGTTTCAGTTGAACTTTGGTCTCTGCGAAGG
GCGAATTCGTTTAAACCTGCAGGACTAGAGGTCCTGTATTAGAGGTCACG
TGAGTGTTTTGCGACATTTTGCGACACCATGTGGTCACGCTGGGTATTTA
AGCCCGAGTGAGCACGCAGGGTCTCCATTTTGAAGCGGGAGGTTTGAACG
CGCAGCCGCCAAGCCGAATTCTGCAGATATCCATCACACTGGCGGCCGCT
CGACTAGAGCGGCCGCCACCGCGGTGGAGCTCCAGCTTTTGTTCCCTTTA
GTGAGGGTTAATTGCGCGCTTGGCGTAATCATGGTCATAGCTGTTTCCTG
TGTGAAATTGTTATCCGCTCACAATTCCACACAACATACGAGCCGGAAGC
ATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAG
AATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAA
ACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTT
TCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTC
CAACCCGGTAAGACACGACTTATCGCCACTGGCAGCAGCCACTGGTAACA
GGATTAGCAGAGCGAGGTATGTAGGCGGTGCTACAGAGTTCTTGAAGTGG
TGGCCTAACTACGGCTACACTAGAAGAACAGTATTTGGTATCTGCGCTCT
GCTGAAGCCAGTTACCTTCGGAAAAAGAGTTGGTAGCTCTTGATCCGGCA
AACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGG
GTCTGACGCTCAGTGGAACGAAAACTCACGTTAAGGGATTTTGGTCATGA
GATTATCAAAAAGGATCTTCACCTAGATCCTTTTAAATTAAAAATGAAGT
TTTAAATCAATCTAAAGTATATATGAGTAAACTTGGTCTGACAGTTACCA
ATGCTTAATCAGTGAGGCACCTATCTCAGCGATCTGTCTATTTCGTTCAT
CCATAGTTGCCTGACTCCCCGTCGTGTAGATAACTACGATACGGGAGGGC
TTACCATCTGGCCCCAGTGCTGCAATGATACCGCGAGACCCACGCTCACC
GGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTT
CATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAG
TAAGTTGGCCGCAGTGTTATCACTCATGGTTATGGCAGCACTGCATAATT
CTCTTACTGTCATGCCATCCGTAAGATGCTTTTCTGTGACTGGTGAGTAC
TCAACCAAGTCATTCTGAGAATAGTGTATGCGGCGACCGAGTTGCTCTTG
CCCGGCGTCAATACGGGATAATACCGCGCCACATAGCAGAACTTTAAAAG
TGCTCATCATTGGAAAACGTTCTTCGGGGCGAAAACTCTCAAGGATCTTA
CCGCTGTTGAGATCCAGTTCGATGTAACCCACTCGTGCACCCAACTGATC
TTCAGCATCTTTTACTTTCACCAGCGTTTCTGGGTGAGCAAAAACAGGAA
GGCAAAATGCCGCAAAAAAGGGAATAAGGGCGACACGGAAATGTTGAATA
CTCATACTCTTCCTTTTTCAATATTATTGAAGCATTTATCAGGGTTATTG
TCTCATGAGCGGATACATATTTGAATGTATTTAGAAAAATAAACAAATAG
GGGTTCCGCGCACATTTCCCCGAAAAGTGCCACCTAAATTGTAAGCGTTA
ATATTTTGTTAAAATTCGCGTTAAATTTTTGTTAAATCAGCTCATTTTTT
AACCAATAGGCCGAAATCGGCAAAATCCCTTATAAATCAAAAGAATAGAC
CGAGATAGGGTTGAGTGTTGTTCCAGTTTGGAACAAGAGTCCACTATTAA
AGAACGTGGACTCCAACGTCAAAGGGCGAAAAACCGTCTATCAGGGCGAT
GGCCCACTACGTGAACCATCACCCTAATCAAGTTTTTTGGGGTCGAGGTG
CCGTAAAGCACTAAATCGGAACCCTAAAGGGAGCCCCCGATTTAGAGCTT
GACGGGGAAAGCCGGCGAACGTGGCGAGAAAGGAAGGGAAGAAAGCGAAA
GGAGCGGGCGCTAGGGCGCTGGCAAGTGTAGCGGTCACGCTGCGCGTAAC
CACCACACCCGCCGCGCTTAATGCGCCGCTACAGGGCGCGTCCCATTCGC
CATTCAGGCTGCGCAACTGTTGGGAAGGGCGATCGGTGCGGGCCTCTTCG
CTATTACGCCAGCTGGCGAAAGGGGGATGTGCTGCAAGGCGATTAAGTTG
GGTAACGCCAGGGTTTTCCCAGTCACGACGTTGTAAAACGACGGCCAGTG
AGCGCGCGTAATACGACTCACTATAGGGCGAATTGGGTACCGGGCCCCCC
CTCGATCGAG (SEQ ID NO: 26).

By “AAV2/10Retro vector” is meant a viral vector comprising an AAV10Retro capsid polypeptide or fragment thereof and that transfects a cell of the central nervous system. In some embodiments, the cell is a nerve cell, an astrocyte, a pericyte, a microglial cell, an oligodendrocyte, or a combination thereof. In some embodiments, the nerve cell is a projection neuron. In various embodiments, the AAV2/10Retro vector targets a projection neuron for retrograde infection. In some embodiments, the AAV2/10Retro vector selectively targets damaged neurons where the damage in some embodiments is caused by a traumatic insult. In some embodiments, the AAV2/10Retro vector crosses the blood brain barrier (BBB) or the blood spinal cord barrier (BSCB) at site where neurons are damaged and/or where the BBB or BSCB is damaged, optionally where the damage is caused by a traumatic insult. The sequence of an exemplary plasmid encoding an AAV2/10Retro vector is provided below. In various embodiments, the plasmid encoding the AAV2/10Retro vector comprises a nucleotide sequence with about or at least about 80%, 85%, 90%, 95%, 96%, 97%, 98%, 99%, or 100% identity to the exemplary AAV2/10Retro vector-encoding plasmid sequence provided below. In various embodiments, the AAV2/9Retro vector encodes an AAV2/10Retro virus.

AAV2/10Retro vector-encoding plasmid sequence:

ATGCCGGGGTTTTACGAGATTGTGATTAAGGTCCCCAGCGACCTTGACGA
GCATCTGCCCGGCATTTCTGACAGCTTTGTGAACTGGGTGGCCGAGAAGG
AATGGGAGTTGCCGCCAGATTCTGACATGGATCTGAATCTGATTGAGCAG
GCACCCCTGACCGTGGCCGAGAAGCTGCAGCGCGACTTTCTGACGGAATG
GCGCCGTGTGAGTAAGGCCCCGGAGGCTCTTTTCTTTGTGCAATTTGAGA
AGGGAGAGAGCTACTTCCACATGCACGTGCTCGTGGAAACCACCGGGGTG
AAATCCATGGTTTTGGGACGTTTCCTGAGTCAGATTCGCGAAAAACTGAT
TCAGAGAATTTACCGCGGGATCGAGCCGACTTTGCCAAACTGGTTCGCGG
TCACAAAGACCAGAAATGGCGCCGGAGGCGGGAACAAGGTGGTGGATGAG
TGCTACATCCCCAATTACTTGCTCCCCAAAACCCAGCCTGAGCTCCAGTG
GGCGTGGACTAATATGGAACAGTATTTAAGCGCCTGTTTGAATCTCACGG
AGCGTAAACGGTTGGTGGCGCAGCATCTGACGCACGTGTCGCAGACGCAG
GAGCAGAACAAAGAGAATCAGAATCCCAATTCTGATGCGCCGGTGATCAG
ATCAAAAACTTCAGCCAGGTACATGGAGCTGGTCGGGTGGCTCGTGGACA
AGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTT
GGACAATGCGGGAAAGATTATGAGCCTGACTAAAACCGCCCCCGACTACC
TGGTGGGCCAGCAGCCCGTGGAGGACATTTCCAGCAATCGGATTTATAAA
ATTTTGGAACTAAACGGGTACGATCCCCAATATGCGGCTTCCGTCTTTCT
GGGATGGGCCACGAAAAAGTTCGGCAAGAGGAACACCATCTGGCTGTTTG
GGCCTGCAACTACCGGGAAGACCAACATCGCGGAGGCCATAGCCCACACT
GTGCCCTTCTACGGGTGCGTAAACTGGACCAATGAGAACTTTCCCTTCAA
CGACTGTGTCGACAAGATGGTGATCTGGTGGGAGGAGGGGAAGATGACCG
CCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGAT
CGTCACCTCCAACACCAACATGTGCGCCGTGATTGACGGGAACTCAACGA
CCTTCGAACACCAGCAGCCGTTGCAAGACCGGATGTTCAAATTTGAACTC
ACCCGCCGTCTGGATCATGACTTTGGGAAGGTCACCAAGCAGGAAGTCAA
AGACTTTTTCCGGTGGGCAAAGGATCACGTGGTTGAGGTGGAGCATGAAT
TCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGAC
GTCAGACGCGGAAGCTTCGATCAACTACGCAGACAGGTACCAAAACAAAT
GTTCTCGTCACGTGGGCATGAATCTGATGCTGTTTCCCTGCAGACAATGC
GAGAGAATGAATCAGAATTCAAATATCTGCTTCACTCACGGACAGAAAGA
CTGTTTAGAGTGCTTTCCCGTGTCAGAATCTCAACCCGTTTCTGTCGTCA
AAAAGGCGTATCAGAAACTGTGCTACATTCATCATATCATGGGAAAGGTG
CCAGACGCTTGCACTGCCTGCGATCTGGTCAATGTGGATTTGGATGACTG
CATCTTTGAACAATAAATGATTTAAATCAGGTATGGCTGCCGATGGTTAT
CTTCCAGATTGGCTCGAGGACAACCTCTCTGAGGGCATTCGCGAGTGGTG
GGACTTGAAACCTGGAGCCCCGAAACCCAAAGCCAACCAGCAAAAGCAGG
ACGACGGCCGGGGTCTGGTGCTTCCTGGCTACAAGTACCTCGGACCCTTC
AACGGACTCGACAAGGGGGAGCCCGTCAACGCGGCGGACGCAGCGGCCCT
CGAGCACGACAAGGCCTACGACCAGCAGCTCAAAGCGGGTGACAATCCGT
ACCTGCGGTATAACCACGCCGACGCCGAGTTTCAGGAGCGTCTGCAAGAA
GATACGTCTTTTGGGGGCAACCTCGGGCGAGCAGTCTTCCAGGCCAAGAA
GCGGGTTCTCGAACCTCTCGGTCTGGTTGAGGAAGGCGCTAAGACGGCTC
CTGGAAAGAAGAGACCGGTAGAGCCATCACCCCAGCGTTCTCCAGACTCC
TCTACGGGCATCGGCAAGAAAGGCCAGCAGCCCGCGAAAAAGAGACTCAA
CTTTGGGCAGACTGGCGACTCAGAGTCAGTGCCCGACCCTCAACCAATCG
GAGAACCCCCCGCAGGCCCCTCTGGTCTGGGATCTGGTACAATGGCTGCA
GGCGGTGGCGCTCCAATGGCAGACAATAACGAAGGCGCCGACGGAGTGGG
TAGTTCCTCAGGAAATTGGCATTGCGATTCCACATGGCTGGGCGACAGAG
TCATCACCACCAGCACCCGAACCTGGGCCCTCCCCACCTACAACAACCAC
CTCTACAAGCAAATCTCCAACGGGACTTCGGGAGGAAGCACCAACGACAA
CACCTACTTCGGCTACAGCACCCCCTGGGGGTATTTTGACTTTAACAGAT
TCCACTGCCACTTCTCACCACGTGACTGGCAGCGACTCATCAACAACAAC
TGGGGATTCCGGCCCAAGAGACTCAACTTCAAGCTCTTCAACATCCAGGT
CAAGGAGGTCACGCAGAATGAAGGCACCAAGACCATCGCCAATAACCTTA
CCAGCACGATTCAGGTCTTTACGGACTCGGAATACCAGCTCCCGTACGTC
CTCGGCTCTGCGCACCAGGGCTGCCTGCCTCCGTTCCCGGCGGACGTCTT
CATGATTCCTCAGTACGGGTACCTGACTCTGAACAATGGCAGTCAGGCCG
TGGGCCGTTCCTCCTTCTACTGCCTGGAGTACTTTCCTTCTCAAATGCTG
AGAACGGGCAACAACTTTGAGTTCAGCTACCAGTTTGAGGACGTGCCTTT
TCACAGCAGCTACGCGCACAGCCAAAGCCTGGACCGGCTGATGAACCCCC
TCATCGACCAGTACCTGTACTACCTGTCTCGGACTCAGTCCACGGGAGGT
ACCGCAGGAACTCAGCAGTTGCTATTTTCTCAGGCCGGGCCTAATAACAT
GTCGGCTCAGGCCAAAAACTGGCTACCCGGGCCCTGCTACCGGCAGCAAC
GCGTCTCCACGACACTGTCGCAAAATAACAACAGCAACTTTGCCTGGACC
GGTGCCACCAAGTATCATCTGAATGGCAGAGACTCTCTGGTAAATCCCGG
TGTCGCTATGGCAACCCACAAGGACGACGAAGAGCGATTTTTTCCGTCCA
GCGGAGTCTTAATGTTTGGGAAACAGGGAGCTGGAAAAGACAACGTGGAC
TATAGCAGCGTTATGCTAACCAGTGAGGAAGAAATTAAAACCACCAACCC
AGTGGCCACAGAACAGTACGGCGTGGTGGCCGATAACCTGCAACAGCAAA
ACCTAGCAGACCAAGACTACACAAAAACTGCTGCCGCTCCTATTGTAGGG
GCCGTCAACAGTCAAGGAGCCTTACCTGGCATGGTCTGGCAGAACCGGGA
CGTGTACCTGCAGGGTCCTATCTGGGCCAAGATTCCTCACACGGACGGAA
ACTTTCATCCCTCGCCGCTGATGGGAGGCTTTGGACTGAAACACCCGCCT
CCTCAGATCCTGATTAAGAATACACCTGTTCCCGCGGATCCTCCAACTAC
CTTCAGTCAAGCTAAGCTGGCGTCGTTCATCACGCAGTACAGCACCGGAC
AGGTCAGCGTGGAAATTGAATGGGAGCTGCAGAAAGAAAACAGCAAACGC
TGGAACCCAGAGATTCAATACACTTCCAACTACTACAAATCTACAAATGT
GGACTTTGCTGTTAACACAGATGGCACTTATTCTGAGCCTCGCCCCATCG
GCACCCGTTACCTCACCCGTAATCTGTAATTGCTTGTTAATCAATAAACC
GGTTGATTCGTTTCAGTTGAACTTTGGTCTCTGCGAAGGGCGAATTCGTT
TAAACCTGCAGGACTAGAGGTCCTGTATTAGAGGTCACGTGAGTGTTTTG
CGACATTTTGCGACACCATGTGGTCACGCTGGGTATTTAAGCCCGAGTGA
GCACGCAGGGTCTCCATTTTGAAGCGGGAGGTTTGAACGCGCAGCCGCCA
AGCCGAATTCTGCAGATATCCATCACACTGGCGGCCGCTCGACTAGAGCG
GCCGCCACCGCGGTGGAGCTCCAGCTTTTGTTCCCTTTAGTGAGGGTTAA
TTGCGCGCTTGGCGTAATCATGGTCATAGCTGTTTCCTGTGTGAAATTGT
TATCCGCTCACAATTCCACACAACATACGAGCCGGAAGCATAAAGTGTAA
AGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGA
ATCGGCCAACGCGCGGGGAGAGGCGGTTTGCGTATTGGGCGCTCTTCCGC
TTCCTCGCTCACTGACTCGCTGCGCTCGGTCGTTCGGCTGCGGCGAGCGG
TATCAGCTCACTCAAAGGCGGTAATACGGTTATCCACAGAATCAGGGGAT
AACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAA
GACACGACTTATCGCCACTGGCAGCAGCCACTGGTAACAGGATTAGCAGA
GCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAG
TTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTC
AGTGGAACGAAAACTCACGTTAAGGGATTTTGGTCATGAGATTATCAAAA
AGGATCTTCACCTAGATCCTTTTAAATTAAAAATGAAGTTTTAAATCAAT
CTAAAGTATATATGAGTAAACTTGGTCTGACAGTTACCAATGCTTAATCA
GTGAGGCACCTATCTCAGCGATCTGTCTATTTCGTTCATCCATAGTTGCC
TGACTCCCCGTCGTGTAGATAACTACGATACGGGAGGGCTTACCATCTGG
CCCCAGTGCTGCAATGATACCGCGAGACCCACGCTCACCGGCTCCAGATT
TATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCC
GGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTC
ATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAA
TACGGGATAATACCGCGCCACATAGCAGAACTTTAAAAGTGCTCATCATT
GGAAAACGTTCTTCGGGGCGAAAACTCTCAAGGATCTTACCGCTGTTGAG
ATCCAGTTCGATGTAACCCACTCGTGCACCCAACTGATCTTCAGCATCTT
TTACTTTCACCAGCGTTTCTGGGTGAGCAAAAACAGGAAGGCAAAATGCC
GCAAAAAAGGGAATAAGGGCGACACGGAAATGTTGAATACTCATACTCTT
CCTTTTTCAATATTATTGAAGCATTTATCAGGGTTATTGTCTCATGAGCG
GATACATATTTGAATGTATTTAGAAAAATAAACAAATAGGGGTTCCGCGC
ACATTTCCCCGAAAAGTGCCACCTAAATTGTAAGCGTTAATATTTTGTTA
AAATTCGCGTTAAATTTTTGTTAAATCAGCTCATTTTTTAACCAATAGGC
CGAAATCGGCAAAATCCCTTATAAATCAAAAGAATAGACCGAGATAGGGT
TGAGTGTTGTTCCAGTTTGGAACAAGAGTCCACTATTAAAGAACGTGGAC
TCCAACGTCAAAGGGCGAAAAACCGTCTATCAGGGCGATGGCCCACTACG
TGAACCATCACCCTAATCAAGTTTTTTGGGGTCGAGGTGCCGTAAAGCAC
TAAATCGGAACCCTAAAGGGAGCCCCCGATTTAGAGCTTGACGGGGAAAG
CCGGCGAACGTGGCGAGAAAGGAAGGGAAGAAAGCGAAAGGAGCGGGCGC
TAGGGCGCTGGCAAGTGTAGCGGTCACGCTGCGCGTAACCACCACACCCG
CCGCGCTTAATGCGCCGCTACAGGGCGCGTCCCATTCGCCATTCAGGCTG
CGCAACTGTTGGGAAGGGCGATCGGTGCGGGCCTCTTCGCTATTACGCCA
GCTGGCGAAAGGGGGATGTGCTGCAAGGCGATTAAGTTGGGTAACGCCAG
GGTTTTCCCAGTCACGACGTTGTAAAACGACGGCCAGTGAGCGCGCGTAA
TACGACTCACTATAGGGCGAATTGGGTACCGGGCCCCCCCTCGATCGAGG
TCGACGGTATCGGGGGAGCTCGCAGGGTCTCCATTTTGAAGCGGGAGGTT
TGAACGCGCAGCCGCC (SEQ ID NO: 27).

The term “administering” or “administration” of a vector, particle, or polynucleotide to a subject includes any route of introducing or delivering to a subject the vector, particle, or polynucleotide to perform an intended function thereof. Administration can be carried out by any suitable route, including orally, intranasally, parenterally (intravenously, intramuscularly, intraperitoneally, or subcutaneously), intracranially, topically, or by any method describe herein. Administration includes self-administration and administration by another (e.g., a medical professional).

By “agent” is meant any small molecule chemical compound, antibody, nucleic acid molecule, recombinant adeno-associated virus (rAAV) particle, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

The term “brain cells” as used herein refers to cells comprising the brain, including neurons, astroglia, oligodendroglia, and microglia. Many specific cell types belong to each category. For example, neurons include dopaminergic, cholinergic and glutaminergic neurons, to name only a few.

The term “capsid” refers to a protein shell of a virus. In various embodiments, the capsid encloses a polynucleotide molecule.

The “central nervous system” (CNS) as used herein, refers to any component of the central nervous system including the brain and spinal cord, the cells and extracellular materials and fluids.

In this disclosure, “comprises,” “comprising,” “containing” and “having” and the like can have the meaning ascribed to them in U.S. Pat. law and can mean “includes,” “including,” and the like; “consisting essentially of′ or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “contact” is meant the bringing together of two components to allow for an interaction to take place between the components. In various embodiments, one component is a neuron or a cell and the other component is a cell, a recombinant adeno-associated virus particle (rAAV), a nucleotide molecule, or a combination thereof. In various embodiments, contacting a neuron with a rAAV comprises intravenously administering the rAAV to a subject comprising the neuron so that, through blood circulation, the rAAV may come into physical contact with the neuron.

By “damage” is meant a structural alteration resulting from disease or physical insult in impairment of normal function. In various embodiments, a neuron is damaged. In some embodiments, the structural alteration is an alteration in lipid membrane structure or integrity caused by a physical insult.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, fluorescent polypeptides (e.g., green fluorescent protein (GFP), tandem dimer Tomato protein (tdTomato)), electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include neurodegenerative diseases (e.g., Parkinson’s disease, Alzheimer’s disease, Huntington’s disease, amyotrophic lateral sclerosis (ALS), or motor neuron disease).

By “encapsidated” is meant to be enclosed in a protein shell. In various embodiments, the protein shell comprises a viral capsid protein (e.g., virion protein 1 (VP1), virion protein 2 (VP2), virion protein 3 (VP3), and combinations thereof).

A “therapeutically effective amount” of, e.g. cells, a nucleic acid molecule, or a recombinant adeno-associated virus (rAAV) particles, with respect to the subject method, refers to an amount of the cells, nucleic acid molecule, or rAAV particles which when applied as part of a desired dosage regimen causes an improvement in neuronal function according to clinically acceptable standards. In various embodiments, the desired dosage regimen comprises administering a composition comprising the cells, a nucleic acid molecule, or a recombinant adeno-associated virus (rAAV) particles to a subject once according to an acceptable method for administration, which may comprise injecting the cells, nucleic acid molecule, or rAAV at multiple locations. In some embodiments, an effective amount of a recombinant adeno-associated virus (rAAV) particle is an amount sufficient to infect a target tissue or cell. In some embodiments, an effective amount of an rAAV is an amount sufficient to produce a stable somatic transgenic animal model. The effective amount will depend primarily on factors such as the species, age, weight, health of the subject, and the tissue or cell to be targeted, and may thus vary among animal and tissue. For example, in some embodiments, an effective amount of a recombinant adeno-associated virus particle or vector is generally in the range of from about 1 ml to about 100 ml of a solution containing from about 10⁹ to 10¹⁶ genome copies.

“Injured neuron” refers to any neuron surrounded by a blood brain barrier (BBB) or blood spinal cord barrier (BSCB) that has been rendered locally permeable by being damaged. In various embodiments the blood brain barrier (BBB) or blood spinal cord barrier (BSCB) has been injured via blunt or penetrating trauma. In various embodiments, an axon of the injured neuron is damaged by the blunt or penetrating trauma. An injured neuron in various embodiments is a neuron having suffered an insult resulting in a change, either temporary or permanent, in normal function of the neuron. In some embodiments, the neuron is a projection neuron.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

In general, a “gene” or “open reading frame” is a region of a polynucleotide sequence capable of being transcribed to an RNA that either has a regulatory function, a catalytic function, and/or encodes a protein. An eukaryotic gene typically has introns and exons, which may organize to produce different RNA splice variants that encode alternative versions of a mature protein. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

As used herein, “heterologous” is used to refer to a gene, polynucleotide, or polypeptide experimentally put into a cell or virus particle that does not normally comprise that polynucleotide or polypeptide. In various embodiments “heterologous” is used to refer to a sequence derived from a different cell or virus from that virus or cell into which the sequence has been introduced.

The term “infection” refers to the process by which a virus particle enters a cell and/or inserts its genome within a cell. In various embodiments, a site of infection the site at which a virus enters a cell. In some embodiments, the cell is a neuron and the site of infection is within an axon of the neuron. In some embodiments, the site of infection is at a damaged site within an axon of the neuron.

The terms “isolated,” “purified,” or “biologically pure” refer to material that is free to varying degrees from components which normally accompany the material in the material’s original source or surroundings. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using techniques in analytical chemistry or microscopy, for example, polyacrylamide gel electrophoresis, mass spectroscopy, electron microscopy, or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “nucleotide molecule”, “polynucleotide”, or “nucleic acid sequence” are used interchangeably to refer to a molecule comprising RNA or DNA. In various embodiments, the nucleotide molecule or polynucleotide comprises modified nucleotides (e.g., locked nucleic acids (LNA)). In some embodiments, the nucleotide molecule or polynucleotide comprises RNA and DNA. The sugar backbone of the nucleotide molecule is non-limiting and may comprise ribose, deoxyribose, or various other suitable sugars. In some embodiments, the nucleic acid molecule comprises at least two nucleotides covalently linked together. In some embodiments, the nucleic acid molecule of the present invention is single-stranded. In some embodiments, the nucleic acid molecule is double stranded. In some embodiments, the nucleic acid molecule is triple-stranded. In some embodiments, the nucleic acid molecule comprises phosphodiester bonds. In some embodiments, the nucleic acid molecule comprises a single-stranded or double-stranded deoxyribonucleic acid (DNA) or a single-stranded or double-stranded ribonucleic acid (RNA). In some embodiments, the nucleic acid molecule comprises a nucleic acid analog. In some embodiments, the nucleic acid analog has a backbone, comprising a bond other than and/or in addition to a phosphodiester bond, such as, by non-limiting example, phosphoramide, phosphorothioate, phosphorodithioate or O-methylphophoroamidite linkage. In some embodiments, the nucleic acid analog is selected from a nucleic acid analog with a backbone selected from a positive backbone; a non-ionic backbone and a non-ribose backbone. In some embodiments, the nucleic acid molecule contains one or more carbocyclic sugars. In some embodiments, the nucleic acid molecule comprises modifications of its ribose-phosphate backbone. In some embodiments, these modifications are performed to facilitate the addition of additional moieties, such as labels. In some embodiments, these modifications are performed to increase the stability and half-life of such molecules in physiological environments. In some embodiments, the term “polynucleotide” captures sequences that include any of the known base analogues of DNA and RNA such as, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein, “obtaining” as in “obtaining an agent” includes synthesizing, purchasing, or otherwise acquiring the agent.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is said to be “operably linked to” or “operatively linked to” or “operably associated with” the regulatory elements, meaning that the gene or coding region is controlled or influenced by the regulatory element.

As used herein “pharmaceutically acceptable” means that which is useful in preparing a pharmaceutical composition that is generally safe and non-toxic to a subject. In various embodiments, pharmaceutically acceptable components of a composition include substances acceptable for veterinary use and/or human pharmaceutical use. In some embodiments pharmaceutically-acceptable refers to molecular entities and compositions that do not produce an allergic or similar untoward reaction when administered to a subject.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

As used herein, “proximal” is used to indicate adjacency to or nearly contacting. For example, in various embodiments, an axon or nerve cell body is proximal to a site of injury if the axon or nerve cell body is no more than about 1 mm, 2 mm, 3 mm, 4 mm, 5 mm, 6 mm, 7 mm, 8 mm, 9 mm, 10 mm, 15 mm, 20 mm, 25 mm, 30 mm, 40 mm, or 50 mm from a site of injury or from a center of a site of spinal cord injury, as measured along the injured spinal cord.

As used herein “recombinant” is used to refer to molecules or polypeptides formed by laboratory methods of genetic recombination to bring together genetic material from multiple sources to create sequences not otherwise found in nature.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “retrograde infection” is meant spread of a virus following infection at an axon of a neuron to the cell body of the neuron.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 µg/ml denatured salmon sperm DNA (ssDNA). In a most preferred embodiment, hybridization will occur at 42° C. in 250 mM NaCl, 25 mM trisodium citrate, 1% SDS, 50% formamide, and 200 µg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

“Spinal cord injury” refers to any injury to the spinal cord via blunt or penetrating trauma. Extreme flexion or extension (particularly in the neck) of the spine can result in traction on the spinal cord with subsequent injury and the development of neurologic symptoms. Spinal cord injury (SCI) is an insult to the spinal cord resulting in a change, either temporary or permanent, in its normal motor, sensory, or autonomic function.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “targeted” or “selective” means a higher probability or observable bias for interaction with a cell or molecule comprising a characteristic of interest and less probability or observable bias for interaction with a cell or molecule not comprising the characteristic of interest. In various embodiments, the characteristic of interest is cell damage. In some embodiments, the characteristic is a site of physical trauma to a nerve cell. In some embodiments, the interaction comprises infection of the cell comprising the characteristic of interest.

The terms “transduction” and “transfection” are used interchangeably and refers to the process by which a viral vector, nucleic acid molecule, or a portion thereof is introduced into a cell. In various embodiments, the viral vector, nucleic acid, or a portion thereof is integrated into the genome of a cell infected by a virus (e.g., an adeno-associated virus). In various embodiments, the viral vector or portion thereof comprises a nucleotide sequence encoding a heterologous polypeptide or a portion thereof. In this context, “heterologous peptide” refers to any peptide sequence not naturally encoded by an adeno-associated virus (AAV) genome. In various embodiments, a nerve cell transduced by a viral vector expresses a heterologous polypeptide encapsidated by the viral vector (e.g., an AAV2/9Retro vector or an AAV2/10Retro vector). In certain embodiments, the introduction is by contacting the cell with a recombinant adeno-associated virus (rAAV) particle or vector comprising the nucleic acid molecule (e.g., an expression cassette) that is to be introduced. An adeno-associated virus genome is a genome that is in whole or in part derived from an adeno-associated virus that infects humans and other primates. In various embodiments, the adeno-associated virus (AAV) genome is comprised of single-stranded DNA (ssDNA), either positive- or negative-sensed. The genome can comprise inverted terminal repeats (ITRs) at both ends of the DNA and open reading frames and/or genes.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to an agent that contains or carries modified genetic material and can be used to introduce heterologous genes to a host cell. The vector can be, for example, a virus vector, a virus-like particle, a plasmid, or a cosmid. The vector can be a virus particle. A vector as used herein can be composed of either DNA or RNA. In some embodiments, a vector is composed of DNA. The vector can be an expression vector. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “virus-like particle” refers to a particle comprising a viral capsid that does not encapsidate a viral genome. In some embodiments, the virus-like particle does not encapsidate a polypeptide-encoding polynucleotide. In various embodiments, the viral capsid comprises a AAV9Retro VP1 polypeptide, an AAV10Retro VP1 polypeptide, or fragments or combinations thereof. Methods for preparation of virus-like particles and methods for drug-delivery using virus-like particles are known in the art (see, e.g., Chung, Y., et al., (2020), “Viral nanoparticles for drug delivery, imaging, immunotherapy, and theranostic applications,” Adv Drug Deliv Rev., DOI: 10.18388/abp.2016_1275).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic diagram of an experimental design.

FIG. 2 is an illustration listing tissues targeted by different adeno-associated viruses (AAVs).

FIGS. 3A and 3B present schematics, images, and a plot providing a comparison of the ability of 10 adeno-associated virus (AAV) serotypes to cross the blood spinal cord barrier and transduce cells around a lesion after spinal cord crush. AAV2/7, AAV2/9, and AAV2/10 were most effective to target cells around an injury site. FIG. 3A provides representative images of longitudinal sections of the thoracic spinal cord showing transduced cells after tail vein injection of different serotypes of AAVs expressing CAG-H2B-GFP at 3 h after T8 crush injury. Scale bar: 1 mm. FIG. 3B provides a plot showing quantification of GFP (green fluorescent protein) expression along the longitudinal axis. Throughout the figures “a.u.” indicates “arbitrary unit”. Throughout the figures, the representation AAV#1/#2 indicates an adeno-associated virus (AAV) comprising a replication open reading frame (Rep) from AAV#1 and a capsid open reading frame (Cap) from AAV#2; for example, the notation AAV2/7 indicates an adeno-associated virus (AAV) comprising a Rep derived from AAV2 and a Cap derived from AAV7. Throughout the figures, H2B indicates “histone H2B”, GFP indicates “green fluorescent protein”, and the notation H2B-GFP indicates histone H2B fused to GFP. Throughout the figures, the annotation “Retro” indicates a replication open reading frame (Rep) encoding a capsid virion protein 1 (VP1) peptide comprising the amino sequence LADQDYTKTA (SEQ ID NO: 3). Throughout the figures, AAV2/retro is used as an abbreviated form of the notation AAV2/2Retro. Throughout the figures, the notation T1, T2, etc. indicates sequentially numbered thoracic vertebrae and the notation L1, L2, etc. indicates sequentially numbered lumbar vertebrae.

FIGS. 4A and 4B are images and plots demonstrating a time window for adeno-associated viruses (AAVs) entering an injured spinal cord. The images and plots of FIGS. 4A and 4B present the tropism of intravenously injected AAVs in the injured spinal cord acutely following T8 crush injury. FIG. 4A provides images of longitudinal sections of the spinal cord infected with AAV2/9-CAG-H2B-GFP or AAV2/7-CAG-H2B-GFP through tail vein injection before, or 3 h, 24 h, 3 d, or 7 d after a T8 crush. Scale bar: 1 mm. FIG. 4B provides plots showing Quantification of GFP expression in AAV2/9 and AAV2/7 groups.

FIG. 5 provides images showing labeling of long-projecting propriospinal neurons into the thoracic spinal cord. Adeno-associated viruses (AAVs) not comprising a replication open reading frame (Rep) encoding a capsid virion protein 1 (VP1) peptide comprising the amino acid sequence LADQDYTKTA (SEQ ID NO: 3) failed to transduce projection neurons in the brain. The top panel of FIG. 5 provides a representative image showing the expression of H2B-GFP (cell bodies only) in different spinal levels. Scale bar: 1 mm. The bottom panel of FIG. 5 provides Representative image showing the expression of ChR2-tdTomato (cell bodies and axons) in different spinal levels. Scale bar: 1 mm. In FIG. 5 “syn” stands for the synapsin promoter and ChR2-tdTomato indicates channelrhodopsin fused with Tandem dimer Tomato fluorescent protein.

FIG. 6 is a structural image showing the location of the insertion of the amino acid sequence LADQDYTKTA (SEQ ID NO: 3) into the virion protein 1 (VP1) of AAV2/2Retro. In the experiments described herein, this sequence was introduced into VP1 of AAV2/9 and AAV2/10.

FIGS. 7A and 7B are a schematic and a plasmid map.

FIG. 8 is a plasmid map.

FIG. 9 is a plasmid map.

FIGS. 10A and 10B are histological fluorescent images. FIG. 10A is a histological fluorescent image of a crush site. FIG. 10B is a histological fluorescent image of the cortex. In general, throughout the figures, tdTomato (tandem dimer Tomato) fluorescent signals have a slightly darker shade of grey than green fluorescent protein (GFP) fluorescent signals; for example, the bright regions (i.e., light grey) in FIG. 10B correspond to GFP fluorescent signals. AAV2/9Retro transduced injured projection neurons, whereas AAV2/9 did not.

FIGS. 11A and 11B present histological fluorescent images. FIG. 11A is a histological fluorescent image of a T8 crush site. FIG. 11B presents histological fluorescent images of a mouse brain.

FIG. 12 is a histological fluorescent image of a mouse brain.

FIG. 13 is a histological fluorescent images of a spinal cord injection site.

FIG. 14 is a histological fluorescent image of a spinal cord injection site.

FIGS. 15A and 15B are histological fluorescent images of retrograde labeled neurons in the gigantocellular reticular nucleus (Gi).

FIG. 16 is individual (left and middle panels) and overlaid (right panel) histological fluorescent images of retrograde labeled neurons in the gigantocellular reticular nucleus (Gi).

FIGS. 17A and 17B are histological fluorescent images of retrograde labeled neurons in the gigantocellular reticular nucleus (Gi).

FIGS. 18A and 18B are histological fluorescent images of retrograde labeled neurons in sublaterodorsal tegmental nucleus (SLD) / locus coeruleus (LC) and the caudal pontine reticular nucleus (PnC).

FIG. 19 is individual (left and middle panels) and overlaid (right panel) histological fluorescent images of retrograde labeled neurons in sublaterodorsal tegmental nucleus (SLD) /locus coeruleus (LC) and the caudal pontine reticular nucleus (PnC).

FIGS. 20A and 20B are histological fluorescent images of retrograde labeled neurons in the pontine reticular formation (PnO).

FIGS. 21A and 21B are histological fluorescent images of retrograde labeled neurons in the cortex.

FIGS. 22A to 22D are histological fluorescent images of retrograde labeled neurons in sub-cortical regions. FIGS. 22A and 22B are histological fluorescent images of retrograde labeled neurons in hypothalamic nuclei. FIGS. 22C and 22D are histological fluorescent images of retrograde labeled neurons in the red nucleus.

### DETAILED DESCRIPTION OF THE INVENTION

The invention features vectors, compositions, and methods that are useful for transducing neurons with damaged axons.

The invention is based, at least in part, upon the discovery that the viral vectors disclosed herein can be efficiently uptaken by axonal terminals and retrogradely target those axotomized neurons selectively. The viral vectors are useful for the delivery of therapeutic proteins for the treatment of traumatic CNS injuries and possibly neurological diseases, as well as for neuronal imaging. Accordingly, the invention provides vectors that can efficiently cross the blood spinal cord barrier (BSCB) or a blood brain barrier (BBB), wherein the BSCB or the BBB has been damaged and/or otherwise compromised. The adeno-associated virus (AAV) vectors can non-invasively target injured neurons. In some embodiments, the targeted neuron is a projection neuron.

### Recombinant Adeno-Associated Viruses (rAAV)

Adeno-associated virus is a small (20-26 nm), icosahedral, and nonenveloped virus. AAV particles contain a single-stranded DNA genome consisting of approximately 4.7 kb. The genome contains three open reading frames (ORFs) encoding for replication proteins (Rep), capsid proteins (Caps), and the assembly activating protein (AAP), and is flanked by two inverted terminal repeats (ITRs). Interestingly, adeno-associated virus (rAAV) particles have tissue-specific targeting capabilities, such that a heterologous gene of the rAAV will be delivered specifically to one or more predetermined tissue(s) or cell(s). A capsid protein encoded by the rAAV facilitates the tissue-specific targeting. In various embodiments, the recombinant adeno-associated virus (rAAV) particles disclosed herein are encoded by any one of the vectors and/or polynucleotides described herein or produced by any one of the methods described herein.

More than 30 naturally occurring serotypes of AAV are available and are useful in the particles, vectors, nucleotide molecules, and methods described herein. Many natural variants in the adeno-associated virus (AAV) capsid exist, allowing identification and use of an AAV with properties specifically suited for neural cells as well as other cell types. AAV viruses (i.e., AAV particles) can be engineered by conventional molecular biology techniques, making it possible to optimize these particles for cell specific delivery of the desired nucleic acid sequences, for minimizing immunogenicity, for tuning stability and particle lifetime, for efficient degradation, for accurate delivery to the nucleus, etc.’

The use of adeno-associated viruses (AAVs) is a common mode of exogenous delivery of DNA because AAVs are relatively non-toxic, provide efficient gene transfer, and can be easily optimized for specific purposes. Among the serotypes of AAVs isolated from human or non-human primates (NHP) and well characterized, human serotype 2 is the first AAV that was developed as a gene transfer vector. This serotype has been widely used for efficient gene transfer experiments in different target tissues and animal models. Other AAV serotypes include useful in the vectors and methods of this disclosure include, but are not limited to, AAV1, AAV3, AAV4, AAV5, AAV6, AAV6.2, AAV7, AAV8, AAV9, AAV10, rh.10, rh.39, rh,43, CSP3, and the like (see, e.g., WO 2005/033321 and U.S. Pat. No. 7,198,951 for a discussion of various AAV serotypes). In certain embodiments the serotype is selected to optimize a desired mode of delivery. As demonstrated herein AAV7, AAV9, and AAV10 capsids can facilitate targeted crossing of the blood-brain barrier (BBB) or the blood spinal cord barrier (BSCB) at a site of nerve cell injury or damage following intravenous administration to transduce a neuron.

Adeno-associated virus components suitable for inclusion in particles and vectors of the present invention include the capsid proteins, including the virion particle (VPs) proteins VP1, VP2, VP3, and hypervariable regions, the replication proteins (rep), including rep 78, rep 68, rep 52, and rep 40, and the sequences encoding these proteins. These components may be readily utilized in a variety of vector systems and cells. Such components maybe used alone or in combination with other adeno-associated virus (AAV) serotype sequences or fragments, or in combination with elements from other AAV or non-AAV viral sequences. For example, in some embodiments a vector, nucleotide molecule, or particle of the present invention can comprise an AAV2 replication open reading frame (AAV2 Rep) in combination with an AAV7Retro, AAV9Retro, or AAV10Retro capsid open reading frame (AAV7Retro Cap, AAV9Retro Cap, and AAV10Retro Cap, respectively). Where, “Retro” indicates a capsid open reading frame (Cap) encoding a virion protein 1 (VP1) peptide comprising an amino acid sequence with about or at least about 80%, 85%, 90%, 95%, 96%, 97%, 98%, 99%, or 100% identity to LADQDYTKTA (SEQ ID NO: 3). In some embodiments, a retro-AAV2 (Tervo et al., Neuron 92: 372-382, 2016, which is incorporated herein by reference) comprises the amino acid sequence LADQDYTKTA (SEQ ID NO: 3).

In some embodiments, the recombinant adeno-associated virus (rAAV) particle comprises or is a vector of the present invention. In some embodiments, the viral particle is a recombinant AAV particle comprising a nucleic acid comprising a heterologous gene flanked by one or two AAV inverted terminal repeats (ITRs). In various embodiments, the heterologous gene is encapsidated in the AAV particle. The AAV particle comprises capsid proteins. In some embodiments, the vector comprises a heterologous gene operatively linked to control sequences including promoters and transcription initiation and termination sequences, thereby forming an expression cassette.

### Polynucleotide Molecules

Recombinant adeno-associated virus (rAAV) vectors (alternatively, AAV vectors) of the invention comprise a nucleotide sequence encoding a heterologous gene and any associated regulatory sequences (e.g., a promoter described herein and other control sequences described herein), and 5′ and 3′ adeno-associated virus (AAV) inverted terminal repeats (ITRs). This recombinant adeno-associated virus vector can be packaged into a capsid protein encoded by a capsid open reading frame (Cap) and delivered to a selected target cell (e.g., a damaged neuron). In some embodiments, the heterologous gene is a nucleic acid sequence, heterologous to the vector sequences, which encodes a polypeptide, protein, functional RNA molecule (e.g., siRNA, miRNA, miRNA inhibitor) or other gene product, of interest. The nucleic acid coding sequence in certain embodiments is operatively linked to regulatory components in a manner which permits heterologous gene transcription, translation, and/or expression in a cell of a target tissue.

In some embodiments, the recombinant adeno-associated virus (AAV) vectors of the present invention comprise cis-acting 5′ and 3′ inverted terminal repeat (ITR) sequences described, e.g., by B. J. Carter, in “Handbook of Parvoviruses”, ed., P. Tijsser, CRC Press, pp. 155 168 (1990). The inverted terminal repeat (ITR) sequences can be about 50, 100, 125, 140, 145, or 150 bp in length. The ability to modify these inverted terminal repeat (ITR) sequences is within the skill of the art; see, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996). In various embodiments, a heterologous sequence comprised by a vector of the present invention and associated regulatory elements is flanked by 5′ and 3′ adeno-associated virus (AAV) inverted terminal repeat (ITR) sequences. The adeno-associated virus (AAV) inverted terminal repeat (ITR) sequences may be obtained from any known AAV, including, as non-limiting examples, AAV2, AAV7, AAV9, and AAV10.

In various embodiments, vectors of the present invention also include expression control sequences operably linked to the heterologous gene in a manner which permits transcription, translation and/or expression of the gene in a cell transfected with the vector(s) or infected with a virus particle of the invention. Thus, the present invention in various aspects provides an expression cassette. As used herein, “operably linked” sequences include both expression control sequences that are contiguous with the gene of interest (i.e., act in trans) and expression control sequences that act in trans or at a distance to control the gene of interest.

Expression control sequences include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and sequences that enhance secretion of the encoded product. A great number of expression control sequences, including promoters which are native, constitutive, inducible and/or tissue-specific, are known in the art and are suitable for use in embodiments of the present invention.

In some embodiments of the present invention a polyadenylation sequence can be inserted following a heterologous gene sequence. In various embodiments, the polyadenylation sequence is inserted before a 3′ adeno-associated virus (AAV) inverted terminal repeat (ITR) sequence. A rAAV vector useful in the present invention may also comprise an intron sequence. A non-limiting example of an intron sequence is an intron derived from SV-40, and is referred to as the SV-40 T intron sequence. Vectors of the present invention in various embodiments comprise an internal ribosome entry site (IRES). An IRES sequence is used to produce more than one polypeptide from a single gene transcript. An IRES sequence may be used to produce a protein that includes more than one polypeptide chain.

The precise nature of sequences needed for gene expression in host cells may vary between species, tissues or cell types. In some embodiments, vectors of the present invention comprise 5′ non-transcribed and 5′ non-translated sequences involved with the initiation of transcription and translation respectively of a heterologous gene, such as, to provide non-limiting examples, a TATA box, a capping sequence, a CAAT sequence, an enhancer elements, and the like. In various embodiments, a 5′ non-transcribed sequences can include a promoter region that includes a promoter sequence for transcriptional control of an operably joined gene. In some embodiments, vectors of the present invention include enhancer sequences or upstream activator sequences as desired. The vectors of the invention may optionally include 5′ leader or signal sequences. The choice and design of an appropriate vector is within the ability and discretion of one of ordinary skill in the art.

Examples of suitable promoters include, but are not limited to the retroviral Rous sarcoma virus (RSV) LTR promoter (optionally with the RSV enhancer), the cytomegalovirus (CMV) promoter (optionally with the CMV enhancer) (see, e.g., Boshart et al (1985) Cell, 41:521-530), the SV40 promoter, the dihydrofolate reductase promoter, the β-actin promoter (e.g., chicken β-actin promoter), the phosphoglycerol kinase (PGK) promoter, the EF1α promoter, the CBA promoter, UBC promoter, GUSB promoter, NSE promoter, Synapsin promoter, MeCP2 (methyl-CPG binding protein 2) promoter, GFAP; CBh promoter and the like. Exemplary promoters include, but are not limited to, the MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit β-globin promoter (CAG promoter; Niwa et al., Gene, 1991, 108(2):193-9) and the elongation factor 1-alpha promoter (EF1-alpha) promoter (Kim et al., Gene, 1990, 91(2):217-23 and Guo et al., Gene Ther., 1996, 3(9):802-10). In some embodiments, the promoter comprises a human β-glucuronidase promoter or a cytomegalovirus enhancer linked to a chicken β-actin (CBA) promoter. The promoter can be a constitutive, inducible, or repressible promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (see, e.g., WO 98/10088); the ecdysone insect promoter (see, e.g., No et al, Proc. Natl. Acad. Sci. USA, 93:3346-3351 (1996)), the tetracycline-repressible system (see, e.g., Gossen et al, Proc. Natl. Acad. Sci. USA, 89:5547-5551 (1992)), the tetracycline-inducible system (see, e.g., Gossen et al, Science, 268:1766-1769 (1995), and Harvey et al, Curr. Opin. Chem. Biol., 2:512-518 (1998)), the RU486-inducible system (see, e.g., Wang et al, Nat. Biotech., 15:239-243 (1997) and Wang et al, Gene Ther., 4:432-441 (1997)) and the rapamycin-inducible system (see, e.g., Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for a heterologous gene comprised by the vector will be used. The native promoter may be preferred when it is desired that expression of the heterologous gene should mimic the native expression. The native promoter may be used when expression of the heterologous gene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter expresses the heterologous gene in a brain cell and/or in a cell body disposed in the brain. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, microglial cell, ependymal cell, and/or a Purkinje cell. In some embodiments, the promoter expresses the heterologous gene in a neuron. In some embodiments, the heterologous gene is exclusively expressed in neurons (e.g., expressed in a neuron and not expressed in other cells of the CNS, such as glial cells).

In some embodiments, vectors of the present invention comprise expression control sequences imparting tissue-specific gene expression capabilities. In some cases, the tissue-specific expression control sequences bind tissue-specific transcription factors that induce transcription in a tissue specific manner. Exemplary tissue-specific regulatory sequences include, but are not limited to, the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter; alpha-fetoprotein (AFP) promoter, bone osteocalcin promoter; bone sialoprotein promoter, CD2 promoter; immunoglobulin heavy chain promoter; T cell receptor α-chain promoter, neuronal such as neuron-specific enolase (NSE) promoter, neurofilament light-chain gene promoter, and the neuron-specific vgf gene promoter. In some embodiments, the expression control sequence allows for specific expression in the central nervous system (CNS) or a subset of one or more neurons or other CNS cells.

In some embodiments, one or more binding sites for one or more of miRNAs are incorporated in a heterologous gene of an adeno-associated virus vector, to inhibit the expression of the heterologous gene in one or more tissues of a subject harboring the heterologous gene, e.g., non-central nervous system (CNS) tissues. The skilled artisan will appreciate that miRNA binding sites may be selected to control the expression of a heterologous gene in a tissue-specific manner. In some embodiments, a binding site for a miRNA is in the 3′ UTR of the mRNA.

In some embodiments, a vector of the present invention may comprise a replication open reading frame (Rep) from an adeno-associated virus (AAV) serotype that differs from that serotype which corresponds to a capsid open reading frame (Cap) comprised by the vector. In one embodiment, the Rep and Cap are expressed from separate sources (e.g., separate vectors, or a cell and a vector). In another embodiment, the Rep and Cap are fused in frame to one another to form a chimeric adeno-associated virus (AAV) vector, such as AAV2/7, AAV2/9, or AAV2/10. In some embodiments, an AAV1 Rep is fused in frame to Cap of the AAV serotype selected from the group consisting of AAV2, AAV3, AAV4, AAV5, AAV6, AAV6.2, AAV7, AAV7m8, AAV8, AAV9, AAV10, rh.10, rh.39, rh.43, and CSP3. In some embodiments, an AAV2 Rep is fused in frame to Cap of the AAV serotype selected from the group consisting of AAV1, AAV3, AAV4, AAV5, AAV6, AAV6.2, AAV7, AAV7m8, AAV8, AAV9, AAV10, rh.10, rh.39, rh.43, and CSP3. In some embodiments, an AAV3 Rep is fused in frame to Cap of the AAV serotype selected from the group consisting of AAV1, AAV2, AAV4, AAV5, AAV6, AAV6.2, AAV7, AAV7m8, AAV8, AAV9, AAV10, rh.10, rh.39, rh.43, and CSP3. In some embodiments, an AAV4 Rep is fused in frame to Cap of the AAV serotype selected from the group consisting of AAV1, AAV2, AAV3, AAV5, AAV6, AAV6.2, AAV7, AAV7m8, AAV8, AAV9, AAV10, rh.10, rh.39, rh.43, and CSP3. In some embodiments, an AAV5 Rep is fused in frame to Cap of the AAV serotype selected from the group consisting of AAV1, AAV2, AAV3, AAV4, AAV6, AAV6.2, AAV7, AAV7m8, AAV8, AAV9, AAV10, rh.10, rh.39, rh.43, and CSP3. In some embodiments, an AAV6 Rep protein is fused in frame to a Cap of the AAV serotype selected from the group consisting of AAV1, AAV2, AAV3, AAV4, AAV5, AAV6.2, AAV7, AAV7m8, AAV8, AAV9, AAV10, rh.10, rh.39, rh.43, and CSP3. In some embodiments, an AAV6.2 Rep protein is fused in frame to a Cap of the AAV serotype selected from the group consisting of AAV1, AAV2, AAV3, AAV4, AAV5, AAV6, AAV7, AAV7m8, AAV8, AAV9, AAV10, rh.10, rh.39, rh.43, and CSP3. In some embodiments, an AAV7 Rep protein is fused in frame to a Cap of the AAV serotype selected from the group consisting of AAV1, AAV2, AAV3, AAV4, AAV5, AAV6.2, AAV6.2, AAV8, AAV9, AAV10, rh.10, rh.39, rh.43, and CSP3. In some embodiments, an AAV7m8 Rep protein is fused in frame to a Cap of the AAV serotype selected from the group consisting of AAV1, AAV2, AAV3, AAV4, AAV5, AAV6.2, AAV6.2, AAV7, AAV8, AAV9, AAV10, rh.10, rh.39, rh.43, and CSP3. In some embodiments, an AAV8 Rep protein is fused in frame to a Cap of the AAV serotype selected from the group consisting of AAV1, AAV2, AAV3, AAV4, AAV5, AAV6.2, AAV6.2, AAV7, AAV7m8, AAV9, AAV10, rh.10, rh.39, rh.43, and CSP3. In some embodiments, an AAV9 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV10, rh.10, rh.39, rh.43, and CSP3. In some embodiments, an AAV10 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, rh.39, rh.43, and CSP3. In some embodiments, an AAVrh.39 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, AAV10, rh.10, rh.43, and CSP3. In some embodiments, an AAVrh.43 Rep protein is fused in frame to a Cap of the AAV serotype selected from the group consisting of AAV1, AAV2, AAV3, AAV4, AAV5, AAV6.2, AAV6.2, AAV7, AAV7m8, AAV8, AAV9, AAV10, rh.10, rh.39, and CSP3. In some embodiments, an AAVCSP3 Rep protein is fused in frame to a Cap of the AAV serotype selected from the group consisting of AAV1, AAV2,AAV3, AAV4, AAV5, AAV6.2, AAV6.2, AAV7, AAV7m7, AAV8, AAV9, AAV10, rh.10,rh.39, and rh.43.

### Recombinant Adeno-Associated Virus (rAAV) Particle Preparation

Numerous methods are known in the art for the production of recombinant adeno-associated virus (rAAV) particles and/or vectors, including transfection, stable cell line production, and infectious hybrid virus production systems which include adenovirus-AAV hybrids, herpesvirus-AAV hybrids and baculovirus-AAV hybrids. In various embodiments, rAAV production cultures for the production of rAAV virus particles all include: 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a heterologous gene (such as a therapeutic gene) flanked by at least one adeno-associated virus (AAV) inverted terminal repeat (ITR) sequences; and 5) suitable media and media components to support recombinant adeno-associated virus (rAAV) particle production. Suitable media known in the art may be used for the production of rAAV particles. These media include, without limitation, media produced by Hyclone Laboratories and JRH including Modified Eagle Medium (MEM), Dulbecco’s Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant adeno-associated virus (AAV) particles.

Methods for preparing recombinant adeno-associated virus particles can involve culturing a cell which contains a nucleic acid sequence encoding a recombinant adeno-associated virus rAAV particle. In various embodiments, the nucleic acid sequence can comprise a sequence encoding a capsid protein (e.g., AAV7 Cap, AAV9 Cap, or AAV10 Cap) or a fragment thereof and a functional replication open reading frame (Rep) (e.g., AAV2 Rep). In various embodiments, the cell also comprises nucleotide sequences encoding sufficient helper functions to permit packaging of a recombinant adeno-associated virus vector comprising a nucleotide sequence encoding a heterologous gene sequence into the AAV capsid proteins. In various embodiments, the adeno-associated virus (AAV) vector comprises a nucleotide sequence comprising inverted terminal repeats (ITRs) and a sequence encoding a heterologous gene. In various embodiments, the cell comprises the AAV vector.

In some aspects, a method is provided for producing any recombinant adeno-associated virus (rAAV) particle as described herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV vector comprising a heterologous gene encoding a therapeutic polypeptide and/or nucleic acid as described herein flanked by at least one AAV inverted terminal repeat (ITR), and (iii) an AAV helper function; and (b) recovering the rAAV particles produced by the host cell.

In some embodiments, components to be cultured in the host cell to package a rAAV vector in an AAV capsid may be provided to the host cell in trans. Alternatively, any one or more of the components (e.g., recombinant adeno-associated virus vector, Rep open reading frame, Cap open reading frame, and/or helper functions) may be provided by cell that has been engineered to contain one or more of the components using methods known to those of skill in the art. In some embodiments, the cell will contain the component(s) under the control of a promoter. Non-limiting examples of the promoter include all promoters described herein. The promoter can be an inducible promoter or a constitutive promoter. In still another alternative, a selected stable host cell may contain selected component(s) under the control of a constitutive promoter and other selected component(s) under the control of one or more inducible promoters. The cell can be derived from 293 cells (which contain E1 helper functions under the control of a constitutive promoter), but which comprise Rep and/or Cap open reading frames under the control of inducible promoters.

A recombinant adeno-associated virus (AAV) vector, a Rep open reading frame, a Cap open reading frame, and helper functions useful for producing the recombinant adeno-associated virus (rAAV) particles of the invention may be delivered to a cell using any appropriate genetic element (e.g., a vector). The selected genetic element may be delivered by any suitable method, including those described herein. Methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. In various embodiments, a method for generating rAAV virions is not a limitation on the present invention.

In some embodiments, recombinant adeno-associated virus particles may be produced using the triple transfection method, an embodiment of which is described in U.S. Pat. No. 6,001,650. Briefly, a plasmid containing a replication open reading frame (Rep) and a capsid open reading frame (Cap), along with a helper adenoviral plasmid, may be transfected (e.g., using the calcium phosphate method) into a cell line (e.g., HEK-293 cells), and recombinant adeno-associated virus (rAAV) particles may be collected and optionally purified. In some embodiments, recombinant adeno-associated virus (rAAV) particles are produced by transfecting a cell with a vector or nucleotide molecule describe herein to be packaged into the rAAV particles, a vector encoding adeno-associated virus (AAV) helper function genes (i.e., an AAV helper function vector), and/or a vector encoding accessory function genes (i.e., an accessory functions vector). The AAV helper function genes can include Rep and Cap open reading frames, which can function in trans for productive adeno-associated virus replication and encapsidation. The accessory function vector encodes nucleotide sequences for non-AAV derived viral and/or cellular functions upon which AAV is dependent for replication (i.e., “accessory functions”). The accessory functions include those functions useful for adeno-associated virus replication, including, without limitation, those moieties involved in activation of adeno-associated virus (AAV) gene transcription, stage specific AAV mRNA splicing, AAV DNA replication, synthesis of Cap expression products, and AAV capsid assembly. Viral-based accessory functions can be derived from any of the known helper viruses such as adenovirus, herpesvirus, and vaccinia virus.

Recombinant adeno-associated virus (rAAV) particles can be purified and formulated using standard techniques known in the art.

In various embodiments, a cell comprising a nucleotide sequence or vector of the present invention is a 293 cell or a cell derived from a 293 cell. A non-limiting example of a 293 cell is available from ATCC, catalog number CRL-1573. In some embodiments, the cell is a HeLa, A549, 293, or insect-derived cell (e.g., SF-9). The cell can be a mammalian cell, insect cell, plant cell, bacterial cell, archaeal cell, or a fungal cell (e.g., a yeast cell).

Suitable recombinant adeno-associated virus (rAAV) particle production culture media of the present invention may be supplemented with serum or serum-derived recombinant proteins at a level of 0.5%-20% (v/v or w/v). Alternatively, as is known in the art, recombinant adeno-associated virus (rAAV) particles may be produced in serum-free conditions which may also be referred to as media with no animal-derived products. One of ordinary skill in the art may appreciate that commercial or custom media designed to support production of recombinant adeno-associated virus (rAAV) particles may also be supplemented with one or more cell culture components know in the art, including without limitation glucose, vitamins, amino acids, and or growth factors, in order to increase the titer of rAAV in production cultures.

Recombinant adeno-associated virus (rAAV) particle production cultures can be grown under a variety of conditions (over a wide temperature range, for varying lengths of time, and the like) suitable to the particular host cell being utilized. As is known in the art, rAAV particle production cultures can include attachment-dependent cultures which can be cultured in suitable attachment-dependent vessels such as, for example, roller bottles, hollow fiber filters, microcarriers, and packed-bed or fluidized-bed bioreactors. Recombinant adeno-associated virus (rAAV) particle production cultures may also include suspension-adapted host cells such as HeLa, 293, and SF-9 cells which can be cultured in a variety of ways including, for example, spinner flasks, stirred tank bioreactors, and disposable systems such as the Wave bag system.

Recombinant adeno-associated virus (rAAV) particles of the invention may be harvested from rAAV production cultures by lysis of the host cells of the production culture or by harvest of the spent media from the production culture, provided the cells are cultured under conditions known in the art to cause release of rAAV particles into the media from intact cells. Suitable methods of lysing cells are also known in the art and include for example multiple freeze/thaw cycles, sonication, microfluidization, and treatment with chemicals, such as detergents and/or proteases.

In some embodiments, a production culture harvest is clarified to remove host cell debris. In some embodiments, the production culture harvest is clarified by filtration through a series of depth filters including, for example, a grade DOHC Millipore Millistak+ HC Pod Filter, a grade A1HC Millipore Millistak+ HC Pod Filter, and a 0.2 µm Filter Opticap XL1O Millipore Express SHC Hydrophilic Membrane filter. Clarification can also be achieved by a variety of other standard techniques known in the art, such as, centrifugation or filtration through any cellulose acetate filter of 0.2 µm or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

Recombinant adeno-associated virus (rAAV) particles may be isolated or purified in various embodiments using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. Methods to purify rAAV particles are found, for example, in Xiao et al., (1998) Journal of Virology 72:2224-2232; U.S. Pat. Nos. 6,989,264 and 8,137,948; and WO 2010/148143.

### Polypeptide Expression

In order to express the polypeptides of the invention, DNA molecules obtained by any of the methods described herein or those that are known in the art, can be inserted into appropriate expression vectors by techniques well known in the art. For example, a double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

Therefore, the invention includes vectors (e.g., recombinant plasmids) that include nucleic acid molecules (e.g., genes or recombinant nucleic acid molecules encoding genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a gene, or fragment thereof, operatively linked to regulatory sequences, e.g., promoter sequences, terminator sequences, and the like, as defined herein. Recombinant vectors which allow for expression of the genes or nucleic acids included in them are referred to as “expression vectors.”

In some of the molecules of the invention described herein, one or more DNA molecules having a nucleotide sequence encoding one or more polypeptides of the invention are operatively linked to one or more regulatory sequences, which are capable of integrating the desired DNA molecule into a cell. Cells which have been stably transformed by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use.

It can be advantageous to codon-optimize a nucleotide sequence encoding one or more polypeptides of the invention for expression in a host organism. For example, a nucleotide sequence encoding a polypeptide of the invention can be codon-optimized for expression in a human cell. Also, polypeptide sequences of the invention can be humanized to facilitate expression in a human cell.

Factors of importance in selecting a particular plasmid or viral vector include, but are not limited to, the ease with which recipient cells that contain the vector are recognized and selected from those recipient cells which do not contain the vector; the number of copies of the vector which are desired in a particular host; and whether it is desirable to be able to “shuttle” the vector between host cells of different species.

Once the vector(s) is constructed to include a DNA sequence for expression, it may be introduced into an appropriate host cell by one or more of a variety of suitable methods that are known in the art, including but not limited to, for example, transformation, transfection, conjugation, protoplast fusion, electroporation, calcium phosphate-precipitation, direct microinjection, etc.

After the introduction of one or more vector(s), host cells are usually grown in a selective medium, which selects for the growth of vector-containing cells. Expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

### Pharmaceutical Compositions

In some aspects, the present invention provides pharmaceutical compositions. To prepare the pharmaceutical compositions of this invention, an effective amount of the recombinant adeno-associated virus (rAAV) particles, nucleotide molecules, and/or vectors of the present invention are combined with a pharmaceutically acceptable carrier, which carrier may take a wide variety of forms depending on the form of preparation desired for administration. These pharmaceutical compositions are desirable in unitary dosage form suitable, particularly, for administration percutaneously, or by parenteral injection. Any of the usual pharmaceutical media may be employed such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules, and tablets. For parenteral compositions, the carrier will usually comprise sterile water, at least in large part, though other ingredients, for example, to aid solubility and cell viability, may be included. Other ingredients may include antioxidants, viscosity stabilizers, chelating agents, buffers, preservatives. If desired, further ingredients may be incorporated in the compositions, e.g. anti-inflammatory agents, antibacterials, antifungals, disinfectants, vitamins, antibiotics.

One suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. Supplementary active ingredients can also be incorporated into the compositions.

In some embodiments, the pharmaceutical composition comprises preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, recombinant adeno-associated virus particle compositions are formulated to reduce aggregation of recombinant adeno-associated virus (rAAV) particles in the composition, particularly where high rAAV concentrations are present (e.g., about 10¹³ gc/ml or more). Methods for reducing aggregation of rAAVs are known in the art and, include, for example, addition of surfactants, pH adjustment, salt concentration adjustment, etc.

In some embodiments, the pharmaceutical compositions of the present invention may contain at least about 0.1% of an additional active compound. A concentration of the additional active compound may conveniently be between about 1 or 2% and about 70% or 80% or more of the weight or volume of the total formulation. The amount of active compound in a pharmaceutical composition may be prepared is such a way that a suitable dosage will be obtained in any given unit dose of the pharmaceutical composition. Factors such as solubility, bioavailability, biological half-life, route of administration, product shelf life, as well as other pharmacological considerations will be contemplated by one skilled in the art of preparing such pharmaceutical compositions, and as such, a variety of dosages and treatment regimens may be desirable.

In some embodiments, the pharmaceutical compositions of the present invention comprise an exosome. In some embodiments, the adeno-associated virus (AAV) particles are exosome-associated AAV vectors, as described in Gene. Ther. 23:380-392 (2016), the entirety of the disclosure of which is incorporated herein by reference for all purposes.

The pharmaceutical compositions suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. In various embodiments, compositions of the present invention are stable under conditions of manufacture and storage and are preserved against the contaminating action of microorganisms, such as bacteria and fungi. A carrier suitable for use in the pharmaceutical composition can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In some embodiments, the pharmaceutical composition may comprise isotonic agents, for example, sugars or sodium chloride. Prolonged absorption of an injectable composition can be brought about by the use in the compositions of agents delaying absorption, for example, aluminum monostearate and gelatin.

The pharmaceutical composition may be buffered, if necessary, and a liquid diluent first rendered isotonic with sufficient saline or glucose. For example, a recombinant adeno-associated virus (rAAV) particle, vector, or plasmid may be dissolved or dispersed in 1 ml of isotonic NaCl solution and either added to 1000 ml of hypodermoclysis fluid or injected at a proposed site of administration on a patient.

Sterile injectable solutions can be prepared by incorporating a recombinant adeno-associated virus (rAAV) particle, vector, or plasmid in a solvent with various of the other ingredients enumerated herein, followed by filtered sterilization. Generally, dispersions are prepared by incorporating the various sterilized active ingredients into a sterile vehicle which contains the basic dispersion medium and other ingredients from those enumerated herein. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

Delivery vehicles such as liposomes, nanocapsules, microparticles, microspheres, lipid particles, vesicles, and the like, may be used for the introduction of the compositions of the present invention into target cells. In particular, a recombinant adeno-associated virus (rAAV) particle, vector, or plasmid may be formulated for delivery encapsulated in a lipid particle, a liposome, a vesicle, a nanosphere, or a nanoparticle or the like. Liposomes have been developed with improved serum stability and circulation half-times (see, e.g., U.S. Pat. No. 5,741,516). Further, various methods of liposome and liposome like preparations as potential drug carriers are known to one of skill in the art.

Nanocapsule formulations of the recombinant adeno-associated virus (rAAV) particle, vector, or plasmid may be used. Nanocapsules can generally entrap substances in a stable and reproducible way.

### Method of Treatment

The present invention provides methods of treating disease, injury, and/or disorder or symptoms thereof, where the methods involve administering a therapeutically effective amount of a pharmaceutical composition comprising a vector of the invention to a subject (e.g., a mammal such as a human). The method includes the step of administering to a subject a therapeutic amount of a composition described herein sufficient to treat the disease, injury, or disorder or symptom thereof, under conditions such that the disease or disorder is treated. In some embodiments, the composition is a pharmaceutical composition described herein.

A neuron or the spinal cord can be injured by a trauma. With respect to trauma, trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of the central nervous system (CNS) tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

As disclosed in the Examples provided below, efficiency of infection of a neuron by recombinant adeno-associated virus (rAAV) particles of the present invention decreases with time from incidence of insult or trauma causing damage or injury to the neuron. In various embodiments, the method comprises administering a composition of the present invention and/or a recombinant adeno-associated virus (rAAV) particle of the present invention to a subject within about 1 hr, 2 hr, 3 hr, 4 hr, 5 hr, 6 hr, 7 hr, 8 hr, 9 hr, 10 hr, 11 hr, 12 hr, 24 hr, 48 hr, 72 hr, 4 days, 5 days, 6 days, or 7 days from incidence of a trauma or injury to a subject causing damage to a neuron. In some embodiments, the method comprises administering a composition of the present invention during incidence of an injury to a neuron (e.g., during the ongoing progression or incidence of a disease or disorder causing neuron damage, blood brain barrier damage, and/or blood spinal cord barrier damage).

The subject method has wide applicability to the treatment of central nervous system (CNS) damage. In this regard, the subject method is useful for, but not limited to, treatment of injury to the brain and spinal cord due to ischemias, hypoxia, traumas, neurodegenerative diseases, infectious diseases, cancers, autoimmune diseases and metabolic disorders. Examples of disorders include stroke, aneurism, head trauma, spinal trauma, hypotension, arrested breathing, cardiac arrest, Reye’s syndrome, cerebral thrombosis, embolism, cerebral hemorrhage, brain tumors, encephalomyelitis, hydroencephalitis, and operative and postoperative brain injury Alzheimer’s disease, Huntington’s disease, Creutzfeld-Jakob disease, Parkinson’s disease, multiple sclerosis and amyotrophic lateral sclerosis.

Thrombus, embolus, and systemic hypotension are common causes of cerebral ischemic episodes. Other causes of cerebral ischemia include hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention in general comprise administration of a therapeutically effective amount of the compositions described herein, such as a composition comprising a recombinant adeno-associated virus (rAAV) particle or vector, to a subject (e.g., animal, human) in need thereof, including a mammal, particularly a human. Such treatment will be suitably administered to subjects, particularly humans, suffering from, having, susceptible to, or at risk for a disease, injury, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any injury, disorder, or disease resulting in nerve damage.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. In some embodiments, the administration modalities as described in U.S. Pat. Nos. 5,543,158; 5,641,515 and 5,399,363 may be used to deliver compositions of the present invention. In some embodiments, a preferred mode of administration is by portal vein injection.

For therapeutic uses, the compositions and agents disclosed herein may be administered by any convenient method; for example, parenterally, conveniently in a pharmaceutically or physiologically acceptable carrier, e.g., phosphate buffered saline, saline, deionized water, or the like. The compositions may be added to a retained physiological fluid such as blood or synovial fluid. For central nervous system (CNS) administration, a variety of techniques are available for promoting transfer of the therapeutic across the blood brain barrier including disruption by surgery or injection, drugs which transiently open adhesion contact between central nervous system (CNS) vasculature endothelial cells, and compounds which facilitate translocation through such cells. As examples, many of the disclosed compositions are amenable to be directly injected or infused or contained within implants e.g. osmotic pumps, grafts comprising appropriately transformed cells. Compositions of the present invention may also be amenable to direct injection or infusion, topical, intratracheal/nasal administration e.g. through aerosol, intraocularly, or within/on implants e.g. fibers e.g. collagen, osmotic pumps, or grafts comprising appropriately transformed cells. Generally, the amount administered will be empirically determined. In various embodiments, a dosage of the compositions of the present invention administered to a subject will generally be in the range of about or at least about 1E+9 gc, 1E+10 gc, 1E+11 gc, 1E+12 gc, or 1E+13 gc per kg or total dose. Other additives may be included, such as stabilizers, bactericides, etc. In various embodiments, these additives can be present in conventional amounts.

In various embodiments, the adeno-associated virus (rAAV) particles, nucleotide molecules, and/or vectors of the present invention are administered in sufficient amounts to transfect a cell of a desired tissue (e.g., a damaged neuron, an astrocyte, a pericyte, a microglial cell, or an oligodendrocyte) and to provide sufficient levels of gene transfer and expression without undue adverse effects. Conventional and pharmaceutically acceptable routes of administration include, but are not limited to, direct delivery to a selected organ or tissue (e.g., the spinal cord or brain), oral, inhalation (including intranasal and intratracheal delivery), intraocular, intravenous, intramuscular, subcutaneous, intradermal, intratumoral, and other parental routes of administration. Routes of administration may be combined, if desired.

The dose of adeno-associated virus (rAAV) particles, nucleotide molecules, and/or vectors used to achieve a particular “therapeutic effect,” e.g., the units of dose in genome copies/per kilogram of body weight (gc/kg), will vary based on several factors including, but not limited to: the route of administration, the level of gene or RNA expression used to achieve a therapeutic effect, the specific disease or disorder being treated, and the stability of the gene or RNA product. One of skill in the art can readily determine a dose range to treat a patient having a particular disease, injury, or disorder based on the aforementioned factors, as well as other factors that are well known in the art. In some embodiments, the therapeutic effect is axon regeneration or restoration of neuronal health.

Administration of recombinant adeno-associated virus (rAAV) particles, nucleotide molecules, and/or vectors of the present invention to a subject may be by, for example, intramuscular injection or by administration into the bloodstream of the subject. Administration into the bloodstream may be by injection into a vein, an artery, or any other vascular conduit. In some embodiments, the recombinant adeno-associated virus (rAAV) particles, nucleotide molecules, and/or vectors are administered into the bloodstream by way of isolated limb perfusion, a technique well known in the surgical arts, the method essentially enabling the artisan to isolate a limb from the systemic circulation prior to administration. A variant of the isolated limb perfusion technique, described in U.S. Pat. No. 6,177,403, can also be employed by the skilled artisan to administer the recombinant adeno-associated virus (rAAV) particles, nucleotide molecules, and/or vectors into the vasculature of an isolated limb to potentially enhance transduction into muscle cells or tissue. Moreover, in certain instances, it may be desirable to deliver the virions to the central nervous system (CNS) of a subject. In various embodiments, by “CNS” is meant all cells and tissue of the brain and spinal cord of a vertebrate. Thus, the term can include, but is not be limited to, neuronal cells, glial cells, astrocytes, cereobrospinal fluid (CSF), interstitial spaces, bone, cartilage and the like. Recombinant adeno-associated virus (rAAV) particles, nucleotide molecules, and/or vectors may be delivered directly to the central nervous system (CNS) or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

The compositions of the invention may comprise an recombinant adeno-associated virus (rAAV) particle, nucleotide molecule, and/or vector, either alone or in combination with one or more other recombinant adeno-associated virus (rAAV) particles, nucleotide molecules, and/or vectors (e.g., a second recombinant adeno-associated virus (rAAV) particle, nucleotide molecule, and/or vector encoding one or more different heterologous genes). In some embodiments, a composition comprises 1, 2, 3, 4, 5, 6, 7, 8, 9, 10, or more different recombinant adeno-associated virus (rAAV) particles, nucleotide molecules, and/or vectors each comprising one or more different heterologous genes. In various embodiments, the heterologous gene facilitates repair of an injured axon of a nerve cell. In some embodiments, the heterologous gene encodes insulin-like growth factor 1 (IGF1), phosphatase and tensin homolog (PTEN), suppressor of cytokine signaling 3 (SOCS3), or osteopontin (OPN). In some embodiments, the heterologous gene encodes a dominant negative Nogo receptor, a chemokine GAP43, ATF3, LIF, IL-6, CNTF, an inwardly rectifying K⁺ channel, SDF1/CXCL12, SDF1, KLF4, KLF6, KLF9, Sox11, Lin28, JAK/STAT, Rictor, Raptor, mTOR, c-myc, doublecortin-like kinase 2 (DCLK2), cRheb-1, BDNF, DLK-1, EFA-6, RNA ligase RTCB-1, HIF-1α, c-Jun, Smad1, STAT3, oncomodulin, arginase 1, CAP-23, Sac2, SPRR1a, MAPK, p53, a cytoskeletal component, trans-forming acidic coiled coil (TACC/TAC-1), doublecortin-like-kinase (DCLK/ZYG-8), a SNARE polypeptide (e.g., a v-SNARE), or a combination thereof. In some embodiments, the heterologous gene encodes a cytokine. Heterologous genes suitable for use in the invention include those disclosed in Williams, P., et al., “Axon Regeneration in the Mammalian Optic Nerve”, Annual Review of Vision Science, 6:195-213 (2020); and He, Z. & Yishi J., “Intrinsic Control of Axon Regeneration”, Neuron 90:437-451 (2016), the disclosures of both of which are incorporated herein by reference for all purposes.

Recombinant adeno-associated virus particles, nucleotide molecules, and/or vectors of the present invention can be inserted into a delivery device which facilitates introduction by injection or implantation into a subject. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. Recombinant adeno-associated virus particles, nucleotide molecules, and/or vectors of the invention can be inserted into such a delivery device, e.g., a syringe, in different forms. For example, the recombinant adeno-associated virus particles, nucleotide molecules, and/or vectors can be suspended in a solution or embedded in a support matrix when contained in such a delivery device. As used herein, the term “solution” includes a pharmaceutically acceptable carrier or diluent in which the recombinant adeno-associated virus particles, nucleotide molecules, and/or vectors of the invention remain functional and/or viable. Pharmaceutically acceptable carriers and diluents include saline, aqueous buffer solutions, solvents and/or dispersion media. The use of such carriers and diluents is well known in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. In some embodiments, the selection of the carrier is not a limitation of the present invention. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating recombinant adeno-associated virus particles, nucleotide molecules, and/or vectors as described herein in a pharmaceutically acceptable carrier or diluent and, as other ingredients enumerated herein, followed by filtered sterilization. Optionally, recombinant adeno-associated virus particles, nucleotide molecules, and/or vectors may be administered on support matrices. Support matrices in which recombinant adeno-associated virus particles, nucleotide molecules, and/or vectors can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. These matrices provide support and protection for the cells in vivo.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a bioactive factor at a particular target site.

One feature of certain embodiments of an implant can be the linear release of the recombinant adeno-associated virus particles, nucleotide molecules, and/or vectors, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder, injury, or disease to be treated and the individual patient response. The generation of such implants is generally known in the art.

In another embodiment of an implant recombinant adeno-associated virus particles, nucleotide molecules, and/or vectors are encapsulated in implantable hollow fibers or the like. Such fibers can be pre-spun and subsequently loaded with the recombinant adeno-associated virus particles, nucleotide molecules, and/or vectors, or can be co-extruded with a polymer which acts to form a polymeric coat about the recombinant adeno-associated virus particles, nucleotide molecules, and/or vectors. Such encapsulated cells can then be combined with a neural stimulant.

In addition to the methods of delivery described above, the following techniques are also contemplated as alternative methods of delivering the recombinant adeno-associated virus particles, nucleotide molecules, and/or vectors to a subject. Ultrasound has been used as a device for enhancing the rate and efficacy of drug permeation into and through a circulatory system. Other drug delivery alternatives contemplated are intraosseous injection (see, e.g., U.S. Pat. No. 5,779,708), microchip devices (see, e.g., U.S. Pat. No. 5,797,898), ophthalmic formulations, transdermal matrices (see, e.g., U.S. Pat. Nos. 5,770,219 and 5,783,208), and feedback-controlled delivery (see, e.g., U.S. Pat. No. 5,697,899).

In various embodiments, the viral particle or virus-like particle of the invention has a half-life in the blood a subject of about or of at least about 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 45 min, 1 hour, 1.1 hours, 1.2 hours. 1.3 hours, 1.4 hours, 1.5 hours. 1.6 hours. 1.7 hours. 1.8 hours, 1.9 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, 2.5 days, 3 days, 4 days, 5 days, 6 days, or 7 days. Methods for measuring the half life of an adeno-associated virus (AAV) vector in blood are known in the art (see, e.g., van Gestel, M., et al., “Recombinant Adeno-Associated Virus: Efficient Transduction of the Rat VMH and Clearance from Blood”, PloS One, 9(5): e97639 (2014); doi: 10.1371/journal.pone.0097639).

### Polynucleotide Therapy

In various aspects, the present invention provides for introducing a heterologous gene to a cell. In various embodiments, the cell is a damaged neuron. In some embodiments, the heterologous gene is incorporated into the genome of the damaged neuron. In various embodiments, the heterologous gene is expressed in the damaged neuron and has a therapeutic effect (e.g., restoration or improvement of function to the neuron or reduction of negative consequences of the damage).

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a heterologous protein, variant, or a fragment thereof, can be cloned into an adeno-associated virus vector of the present invention and expression can be driven from a promoter described herein.

In some embodiments, the heterologous gene comprises cDNA. cDNA expression for use in polynucleotide therapy methods can be directed from any suitable promoter (e.g., those described herein) and regulated by any appropriate regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a heterologous gene, regulation can be mediated by the cognate regulatory sequences or, if desired, by regulatory sequences derived from a heterologous source, including any of the promoters or regulatory elements described herein.

In some embodiments, the heterologous gene of the present invention encodes a therapeutic polynucleotide. In some embodiments, a therapeutic nucleic acid may include without limitation an siRNA, an shRNA, an RNAi, a miRNA, an antisense RNA, a ribozyme or a DNAzyme. As such, a therapeutic nucleic acid may encode an RNA that when transcribed from the nucleic acids of a vector of the present invention can treat a disorder or injury (e.g., a disorder of the central nervous system (CNS)); for example, by interfering with translation or transcription of an abnormal or excess protein associated with a disorder. For example, the heterologous gene may encode an RNA which treats a disorder by highly specific elimination or reduction of mRNA encoding abnormal and/or excess proteins. Therapeutic RNA sequences include RNAi, small inhibitory RNA (siRNA), micro RNA (miRNA), and/or ribozymes (such as hammerhead and hairpin ribozymes) that can treat disorders or an injury by highly specific elimination or reduction of mRNA encoding abnormal and/or excess proteins.

In some embodiments, the heterologous gene encodes a therapeutic polypeptide. A therapeutic polypeptide may, e.g., supply a polypeptide and/or enzymatic activity that is absent or present at a reduced level in a cell or organism. Alternatively, a therapeutic polypeptide may supply a polypeptide and/or enzymatic activity that indirectly counteracts an imbalance in a cell or organism. For example, a therapeutic polypeptide for a disorder related to buildup of a metabolite caused by a deficiency in a metabolic enzyme or activity may supply a missing metabolic enzyme or activity, or it may supply an alternate metabolic enzyme or activity that leads to reduction of the metabolite. A therapeutic polypeptide may also be used to reduce the activity of a polypeptide (e.g., one that is overexpressed, activated by a gain-of-function mutation, or whose activity is otherwise mis-regulated) by acting, e.g., as a dominant-negative polypeptide.

In some embodiments, the heterologous gene encodes a polypeptide selected from an enzyme, a neurotrophic factor, a polypeptide that is deficient or mutated in a subject with a CNS-related disorder or neuronal injury, an antioxidant, an anti-apoptotic factor, an anti-angiogenic factor, and an anti-inflammatory factor. Such polypeptides may be used to treat disorders of the CNS by, e.g., supplying a polypeptide and/or enzymatic activity that is reduced, absent, or misregulated in a disorder of the CNS, ameliorating a cause and/or symptom of a CNS disorder, and/or mitigating damage to the CNS caused by a CNS disorder (e.g., apoptosis, inflammation, or other type of cell death). Non-limiting examples of nucleic acid encoding therapeutic polypeptides include: nucleic acids for replacement of a missing or mutated gene known to cause a disorder of the CNS, for example Prph2, RPE65, MERTK, RPGR, RP2, RPGRIP, CNGA3, CNGB3, and GNAT2. Other non-limiting examples of nucleic acids encoding therapeutic polypeptides include those encoding neurotrophic factors (such as GDNF, CNTF, FGF2, PEDF, EPO), anti-apoptotic genes (such as BCL2, BCL-X, NFκB), anti-angiogenic factors (such as Endostatin, Angiostatin, sFlt), and anti-inflammatory factors (such as IL10, IL1-ra, TGFβ, IL4). Other therapeutic polypeptides for CNS disorders include but are not limited to Myo7a, ABCA4, REP1, GUCY2D, PDE6C, RS1, RPGRIP, Lpcat1, AIPL1, RDH12, CHM. In some embodiments, the encoded polypeptide is the human variant of the polypeptide. In some embodiments, the heterologous gene encodes neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxylase (TH), GTP-cyclohydrolase (GTPCH), amino acid decarboxylase (AADC), an anti-oxidant, an anti-angiogenic polypeptide, an anti-inflammatory polypeptide, and/or aspartoacylase (ASPA). Examples of anti-oxidants include without limitation SOD1; SOD2; Catalase; Sirtuins 1, 3, 4, or 5; NRF2; PGC1a; GCL (catalytic subunit); GCL (modifier subunit); adiponectin; glutathione peroxidase 1; and neuroglobin. Examples of anti-angiogenic polypeptides include without limitation angiostatin, endostatin, PEDF, a soluble VEGF receptor, and a soluble PDGF receptor. Examples of anti-inflammatory polypeptides include without limitation IL-10, soluble IL17R, soluble TNF-R, TNF-R-Ig, an IL-1 inhibitor, and an IL18 inhibitor. In some embodiments, the heterologous gene encodes insulin-like growth factor 1 (IGF1), phosphatase and tensin homolog (PTEN), suppressor of cytokine signaling 3 (SOCS3), or osteopontin (OPN). In some embodiments, the heterologous gene encodes a dominant negative Nogo receptor, a chemokine GAP43, ATF3, LIF, IL-6, CNTF, an inwardly rectifying K⁺ channel, SDF1/CXCL12, SDF1, KLF4, KLF6, KLF9, Sox11, Lin28, JAK/STAT, Rictor, Raptor, mTOR, c-myc, doublecortin-like kinase 2 (DCLK2), cRheb-1, BDNF, DLK-1, EFA-6, RNA ligase RTCB-1, HIF-1α, c-Jun, Smad1, STAT3, oncomodulin, arginase 1, CAP-23, Sac2, SPRR1a, MAPK, p53, a cytoskeletal component, trans-forming acidic coiled coil (TACC/TAC-1), doublecortin-like-kinase (DCLK/ZYG-8), a SNARE polypeptide (e.g., a v-SNARE), or a combination thereof. In some embodiments, the heterologous gene encodes a cytokine.

The heterologous genes the invention may encode polypeptides that are intracellular proteins, anchored in the cell membrane, remain within the cell, or are secreted by the cell transduced with the vectors of the invention. For polypeptides secreted by the cell that receives the vector; the polypeptide can be soluble (i.e., not attached to the cell). For example, soluble polypeptides are devoid of a transmembrane region and are secreted from the cell. Techniques to identify and remove nucleic acid sequences which encode transmembrane domains are known in the art.

As described herein, in various embodiments, expression of the heterologous gene can be under the control of a promoter and/or various control sequences.

### Kits

The invention provides kits for the treatment or prevention of a disease, injury, or disorder. The agents described herein may, in some embodiments, be assembled into pharmaceutical or diagnostic or research kits to facilitate their use in therapeutic, diagnostic or research applications. In certain embodiments agents in a kit may be in a pharmaceutical formulation and dosage suitable for a particular application and for a method of administration of the agents. Kits for research purposes may contain the components in appropriate concentrations or quantities for running various experiments (e.g., cell imaging).

Kits may include dose-size-specific ampules or aliquots of compositions of the present invention. Kits may also contain devices to be used in administering the compositions. In some embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit may be designed to facilitate use of the methods described herein. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or another suitable solvent), which may or may not be provided with the kit.

The kit may contain any one or more of the components described herein in one or more containers. As an example, in one embodiment, the kit may include instructions for mixing one or more components of the kit and/or isolating and mixing a sample and administering to a subject. The kit may include a container housing agents described herein. The agents may be in the form of a liquid, gel or solid (powder). The agents may be prepared sterilely, packaged in syringe and shipped refrigerated. A second container may comprise other agents prepared sterilely. Alternatively the kit may include agents premixed and shipped in a syringe, vial, tube, or other container. The kit may have one or more or all of the components useful to administer the agents to a subject, such as a syringe, topical application devices, or intravenous needle tubing and bag.

If desired an agent of the invention is provided together with instructions for administering an agent of the present invention to a subject having or at risk of developing a disease, injury, or disorder described herein. The instructions will generally include information about the use of the composition for the treatment or prevention of the disease, injury, or disorder. In other embodiments, the instructions include at least one of the following: description of the therapeutic agent; dosage schedule and administration for treatment or prevention of a disease, injury, or disorder described herein; precautions; warnings; indications; counter-indications; overdosage information; adverse reactions; animal pharmacology; clinical studies; and/or references. The instructions may be printed directly on the container (when present), provided on a transportable storage medium, stored on a remote server, or provided as a label applied to the container, or as a separate sheet, pamphlet, card, or folder supplied in or with the container. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

In certain aspects, practitioners of the present invention may employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes 1 and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1.: Viral Vectors, After Intravenous Injection, Enter the Spinal Cord Around a Lesion

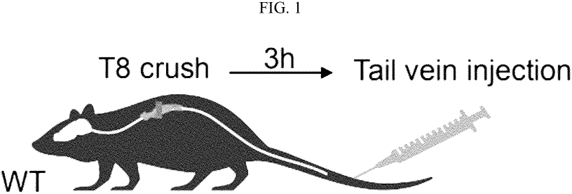

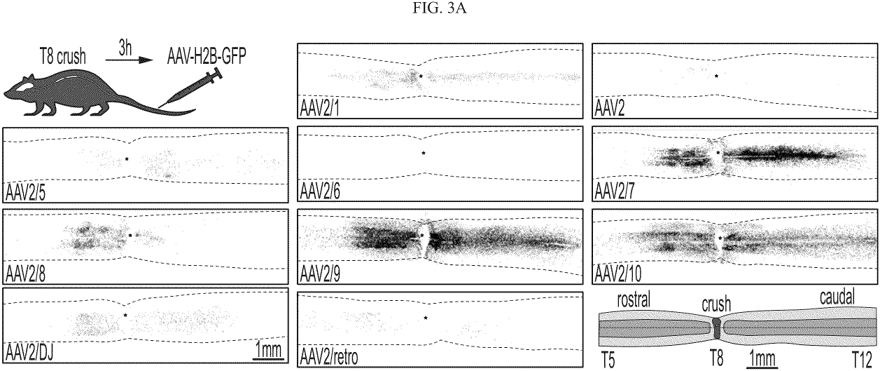

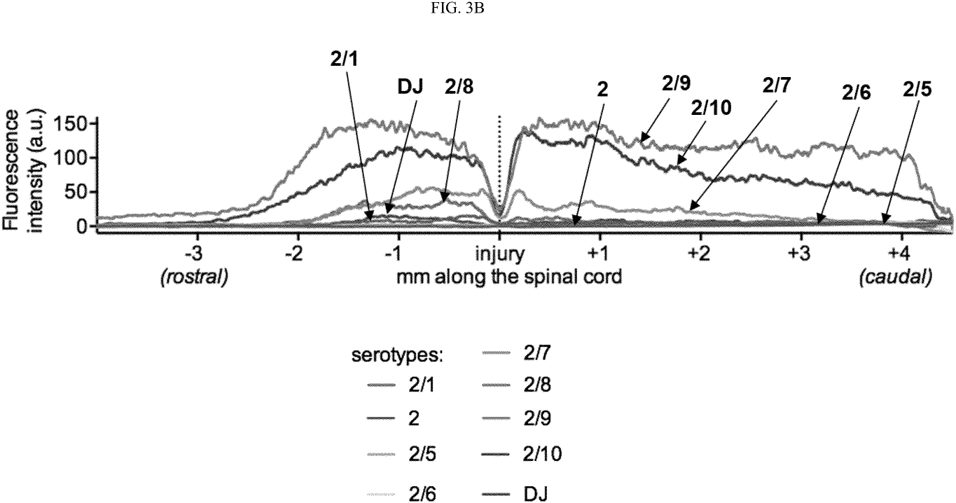

Spinal cord injury can result in the partial or complete loss of sensory and/or motor function below the level of injury. While injured axons lose the ability to regenerate, inflammatory changes in the spinal cord and the formation of a glial scar at the site subsequent to injury create barriers to functional recovery after spinal cord trauma. Gene therapy may provide a means to deliver therapeutic molecules to overcome these barriers and facilitate regeneration. However, identifying vectors capable of crossing the blood brain barrier and being taken up by neurons has proved challenging. In an effort to identify viral vectors that could enter the spinal cord following intravenous injections a series of experiments were undertaken. FIGS. 1 and 3A provide a schematic overview of the experimental design used to identify such vectors.

A murine model of spinal cord injury (SCI) model mice was used. The spinal cord of mice was crushed at the level of T8. The mice were then intravenously injected with a variety of adeno associated viral vectors (Table 1). Successful transduction was assessed using a green fluorescent protein (GFP) reporter construct, where GFP expression was under the control of the cytomegalovirus enhancer/chicken beta-actin/Rabbit β-globin promoter (CAG). CAG is a ubiquitously expressed promoter. GFP was fused to histone 2B (H2B), which localizes to the nuclei of transduced cells, facilitating assessment of transduction efficiency. Adeno-associated viruses evaluated are shown in Table 1.

TABLE 1

Adeno-associated viruses evaluated

| | |
|---|---|
| serotype | AAV Virus |
| AAV1 | AAV2/1-CAG-H2B-GFP |
| AAV2 | AAV2/1-CAG-H2B-GFP |
| AAV5 | AAV2/5-CAG-H2B-GFP |
| AAV6 | AAV2/6-CAG-H2B-GFP |
| AAV7 | AAV2/7-CAG-H2B-GFP |
| AAV8 | AAV2/8-CAG-H2B-GFP |
| AAV9 | AAV2/9-CAG-H2B-GFP |
| AAV10 | AAV2/10-CAG-H2B-GFP |
| AAV2/2Retro | AAV2/2Retro-CAG-H2B-GFP |
| AAV DJ | AAV2/DJ-CAG-H2B-GFP |

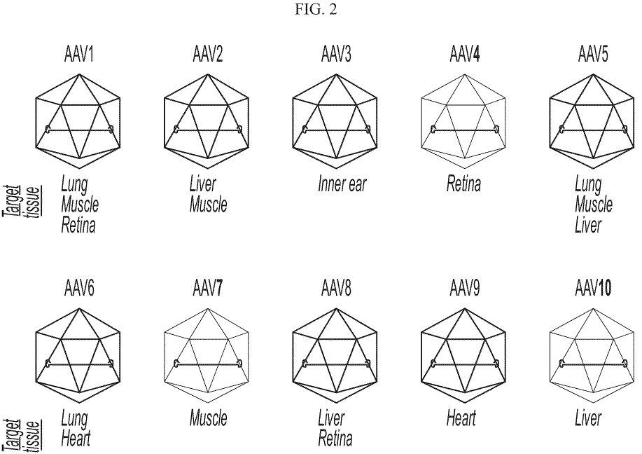

Multiple AAV serotypes exist that transduce a variety of tissues with varying degrees of efficiency. FIG. 2 provides a description of the tissue-tropism of the AAV serotypes analyzed.

Following spinal cord injury, mice were intravenously injected with ten AAV serotypes, and imaging was carried out to determine which AAVs were most effective in targeting cells around an injury site. FIG. 3A depicts the efficacy with which the various AAVs were able to target cells at the injury site (8E12 gc particles dose) when injected into the tail vein of mice 3 hours after a T8 complete crush injury. Expression was measured at 14 days following injection. AAV2/7, 2/9, and 2/10 were most effective in targeting cells at the site of injury, and GFP expression was observed both rostral and caudal to the nerve crush. Throughout the examples, the term AAV#1/#2, where #1 and #2 indicate a first and a second generic number, indicates an adeno-associated virus (AAV) comprising a replication open reading frame (Rep) from AAV#1 (e.g., AAV2)and a capsid open reading frame (Cap) from AAV#2 (e.g., AAV7, AAV9, or AAV10); for example, the notation AAV2/7 indicates an adeno-associated virus (AAV) comprising a replication open reading frame derived from AAV2 and a capsid open reading frame derived from AAV7. FIG. 3B depicts the fluorescence intensity of the various serotypes at distances rostral and caudal to the point of injury.

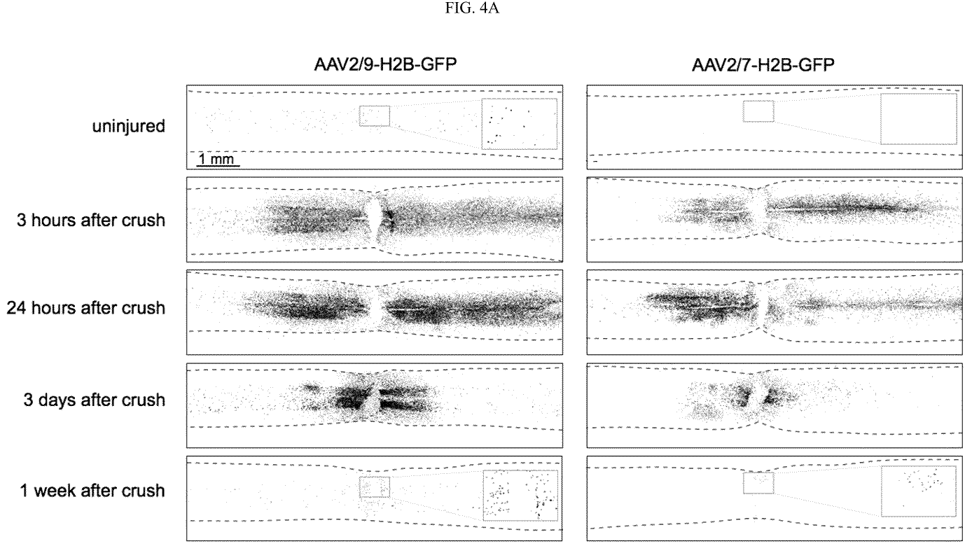

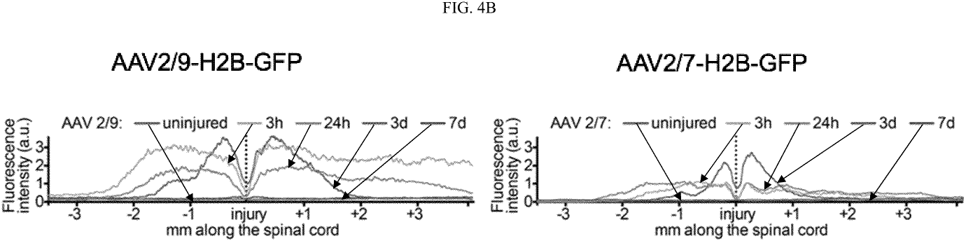

To determine the time points at which AAVs were most efficient in entering the injured spinal cord, AAVs were injected at 3 hours, 24 hours, 3-days, and 1 week after nerve crush. AAV2/9 and AAV2/7 are known to have limited ability of crossing the intact blood brain barrier (BB) in adults. As shown in FIG. 4A, strong GFP expression was detected rostral and caudal to the injury when the AAV vectors were administered within 3 hours to 3-days of the injury. FIG. 4B quantitates the results shown in FIG. 4A, and indicates fluorescence intensity at various distances along the spinal cord.

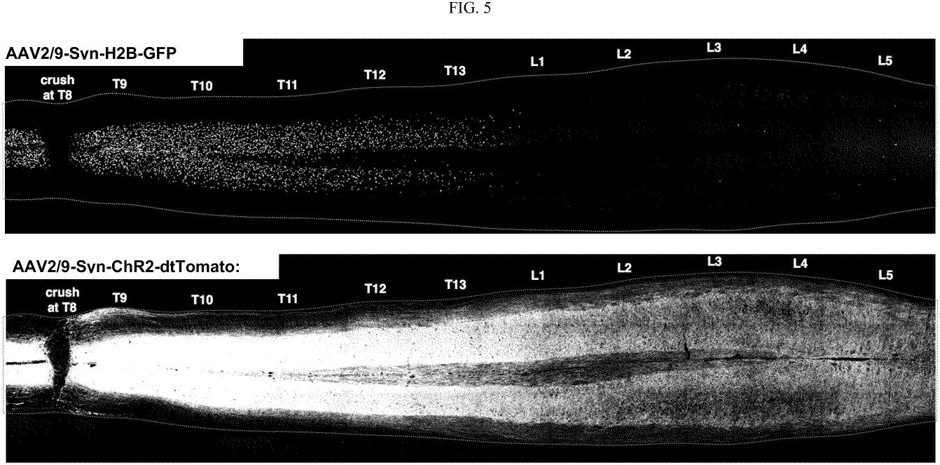

To assess whether cells transduced by the vectors local to the site of injury included propriospinal neurons with descending projections, AAV2/9-Syn-H2B-GFP (to visualize the cell bodies of transduced neurons) and AAV2/9-Syn-ChR2-tdTomato (to visualize both the axons and cell bodies of transduced neurons, including propriospinal axons) were co-injected into the tail vein of adult mice at 3 hours after injury. As shown in the top of FIG. 5, the transduced neuronal somas (GFP+) were almost exclusively localized in thoracic levels around the lesion site. Also, numerous tdTomato+ axons were detected in the lumbar spinal cord (bottom of FIG. 5), suggesting efficient transduction of propriospinal neurons with projections descending into the lumbar spinal cord.

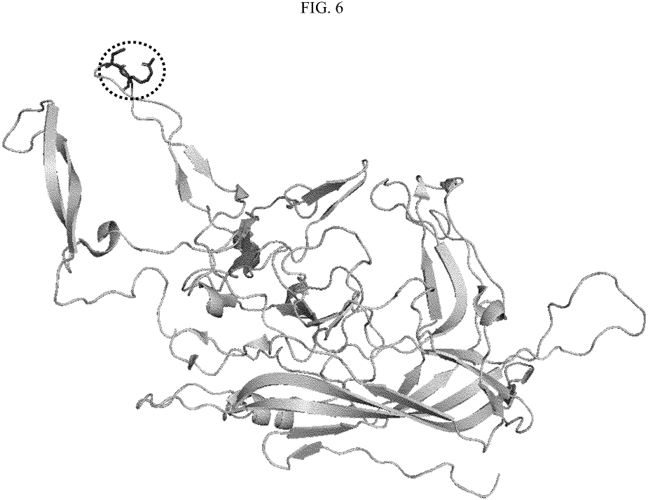

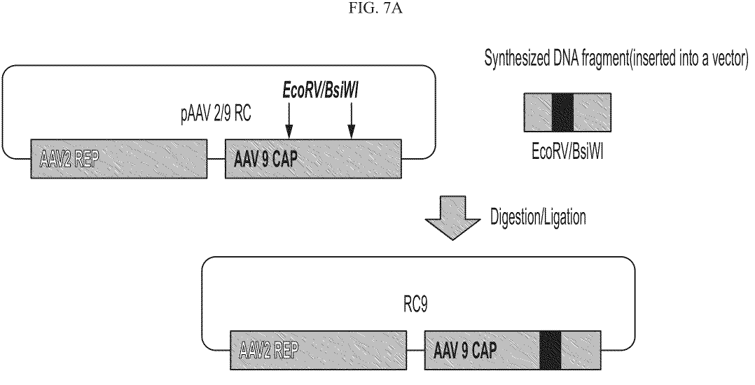

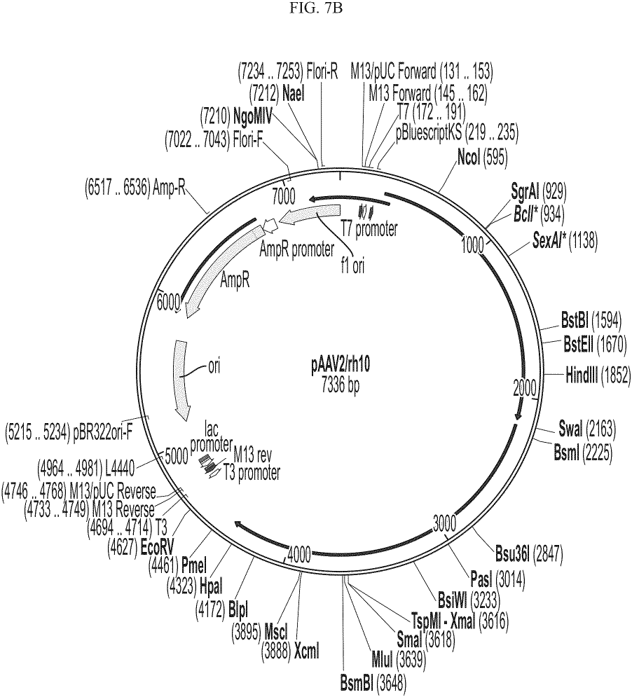

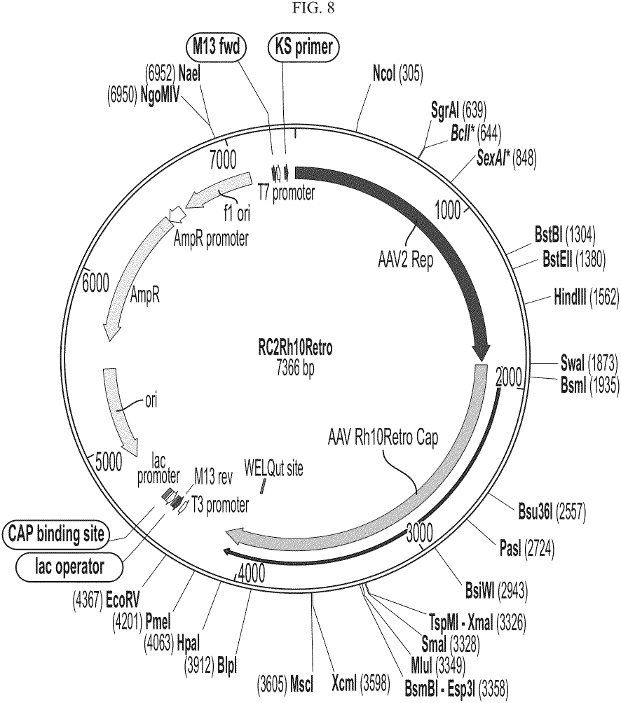

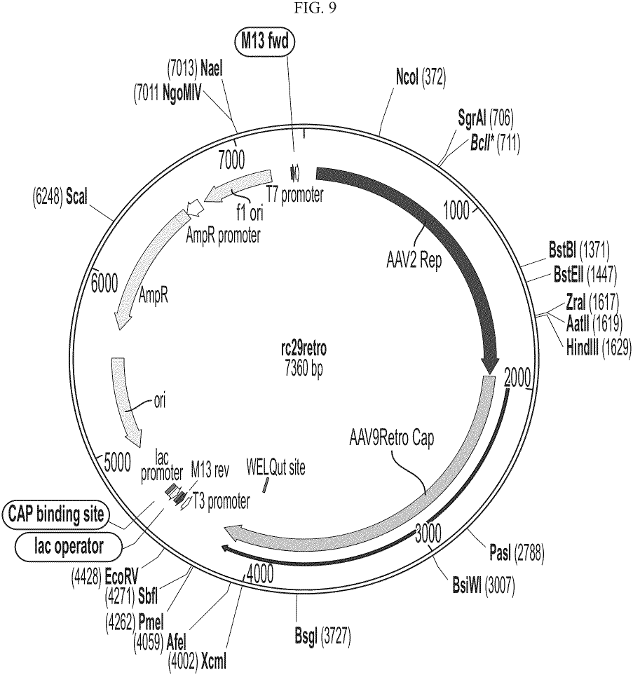

In an effort to obtain an AAV vector capable of crossing the blood brain barrier and having the ability to provide retrograde targeting of projection neurons, thereby transporting the virus and its payload to the nucleus, AAV2/9 was combined with retro-AAV2 (Tervo et al., Neuron 92: 372-382, 2016, which is incorporated herein by reference). Tervo et al. describe the generation of a variant AAV vector, rAAV2-retro, that provides retrograde access to projection neurons, and that includes the amino acid sequence LADQDYTKTA (SEQ ID NO: 3). FIG. 6 provides a structural image showing the location of the amino acid sequence LADQDYTKTA (SEQ ID NO: 3) in the virion protein 1 (VP1) of AAV2/2Retro. The amino acid sequence LADQDYTKTA (SEQ ID NO: 3) was introduced into the virion protein 1 (VP1) peptides of AAV2/9 and AAV2/10 to prepare AAV2/9Retro and AAV2/10Retro, respectively. FIG. 7A provides a schematic summarizing the preparation of an AAV2/9Retro vector-encoding plasmid (pAAV2/9 RC). The dark square depicted in the “synthesized DNA fragment” of FIG. 7A corresponds to a DNA sequence encoding the amino acid sequence LADQDYTKTA (SEQ ID NO: 3). FIGS. 7B and 8 provide vector maps for an AAV2/10Retro vector-encoding plasmid (pAAV2/rh10). FIG. 9 provides a vector map for an AAV2/9Retro vector. Table 2 below provides descriptive information relating to the AAV2/9Retro and AAV2/10Retro vectors.

TABLE 2

Descriptive information relating to the AAV2/9Retro vector-encoding and AAV2/10Retro vector-encoding plasmids.

| | | |
|---|---|---|
| | pAAV2/rh10 (alternatively, RC2Rh10Retro) | AAV2/9 RC (alternatively, rc29retro) |
| PURPOSE | AAV packaging plasmid expressing Rep/Cap genes | AAV packaging plasmid expressing Rep/Cap genes |
| Bacterial Resistance(s) | Ampicillin | Ampicillin |
| Growth Strain(s) | DH5alpha | DH5alpha |
| sequence | 7336 bps | 7360 bps |
| Digestion | ECORV/BSIW1 5944/1392BPS | ECORV/BSIW15941/1389 BPS |

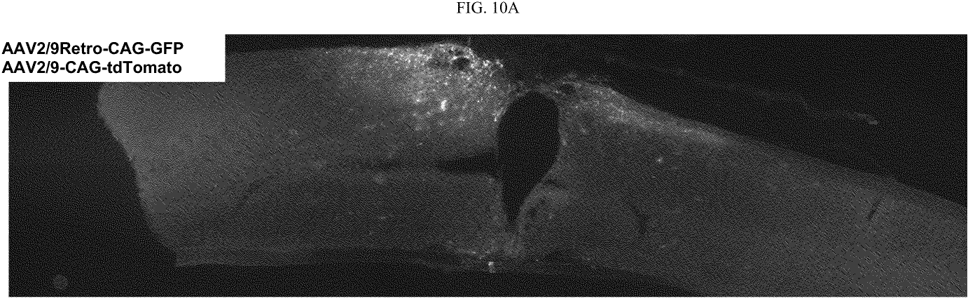

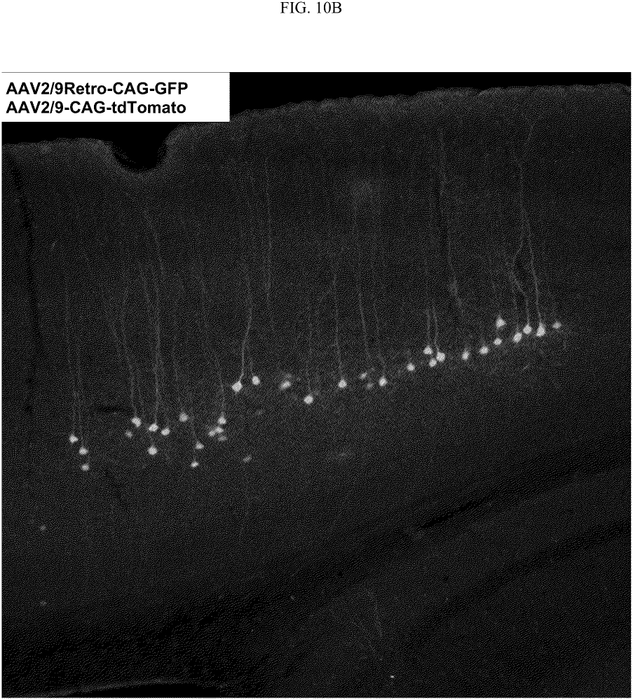

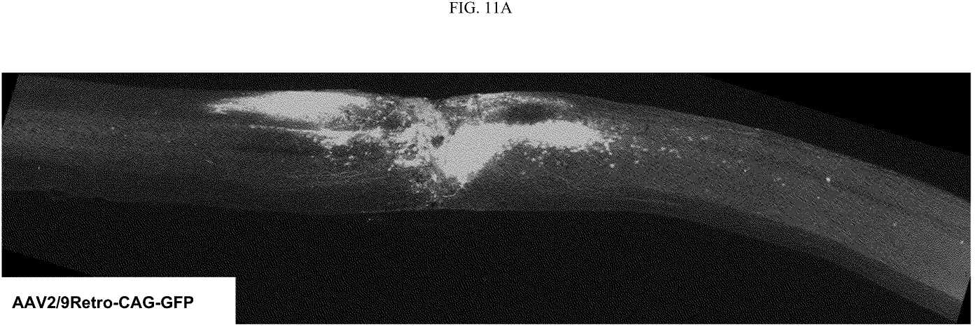

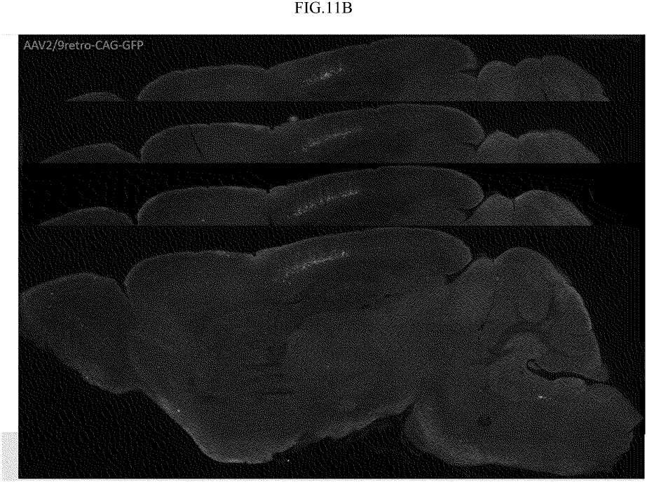

AAV2/10Retro, AAV2/9Retro, and/or AAV2/2Retro were injected systemically via tail vein injection into a murine model of spinal cord injury three hours following nerve crush at thoracic vertebrae T8. 6.43E+11 gc/mouse was used. Expression of a GFP reporter or tdTomato reporter expressed by the vectors was observed for three weeks following administration in projection axons at the crush site (FIGS. 10A, 11A), as well as in neuronal cell bodies present in the cortex (FIGS. 10B, 11B), suggesting that these intravenously injected viral vectors are able to enter injured spinal cord and transduce injured corticospinal neurons.

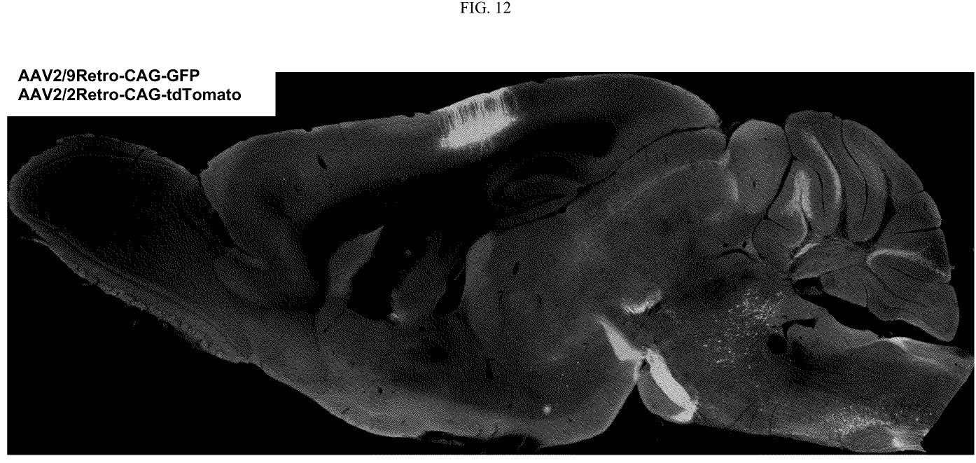

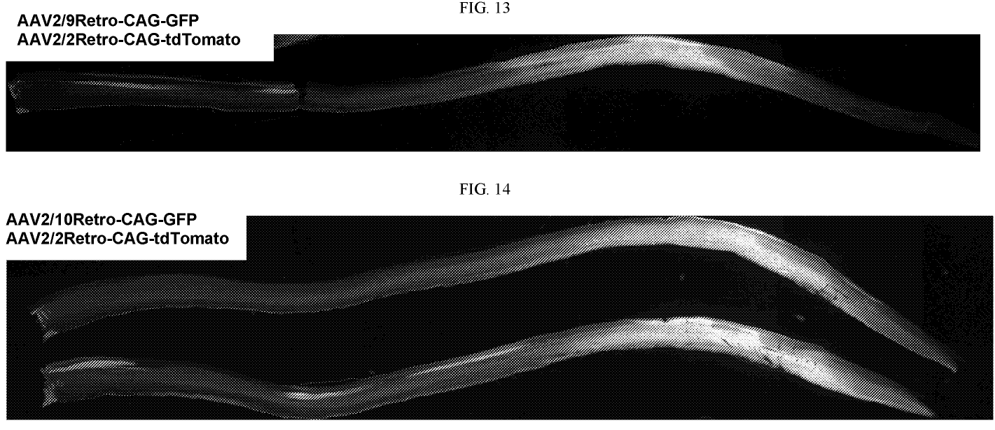

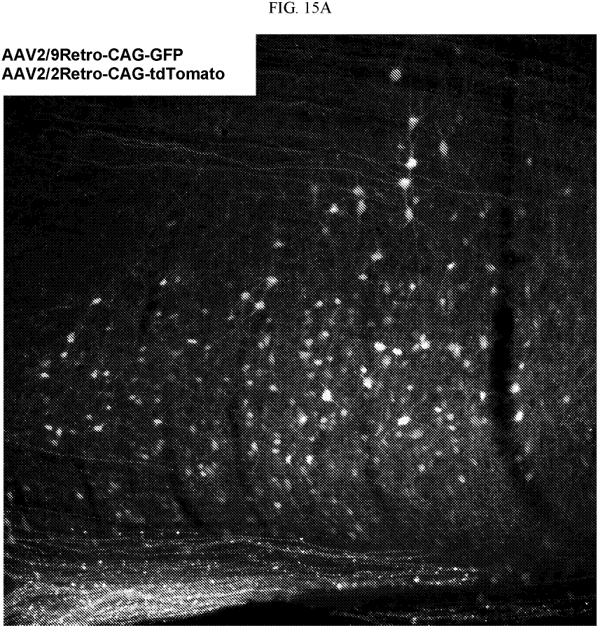

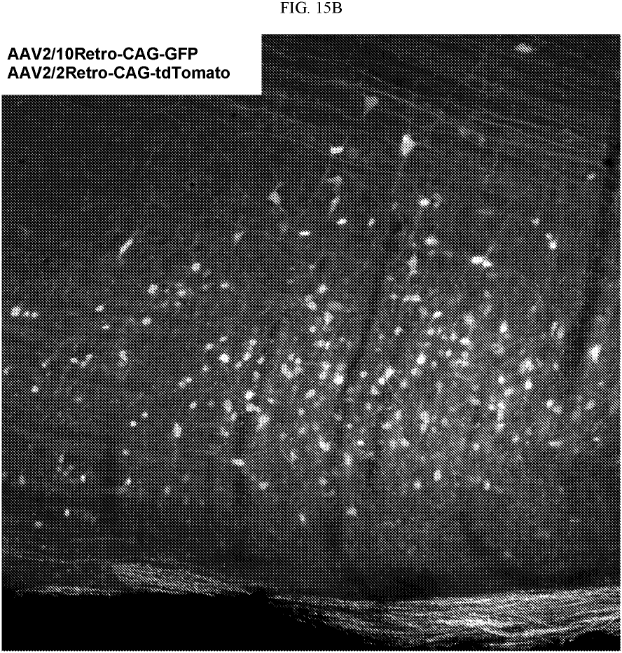

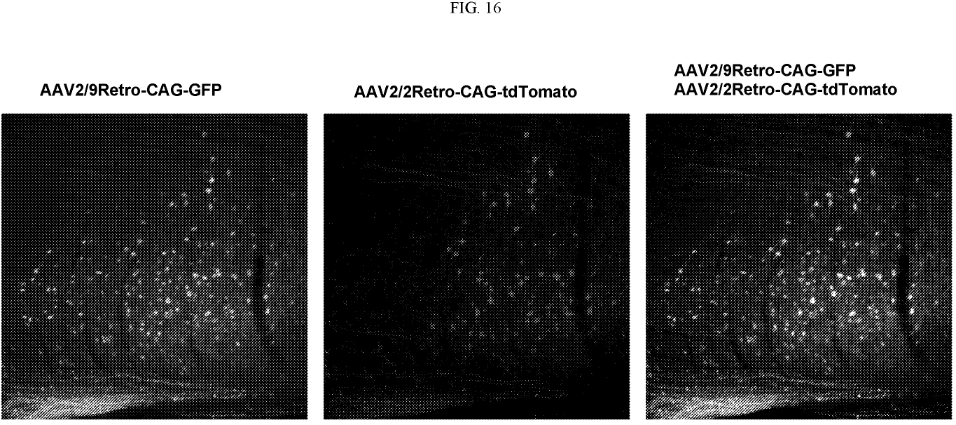

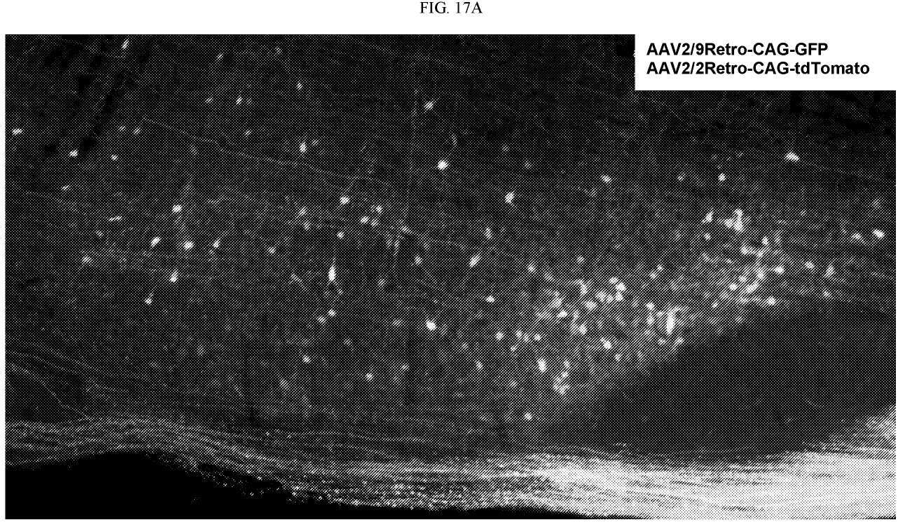

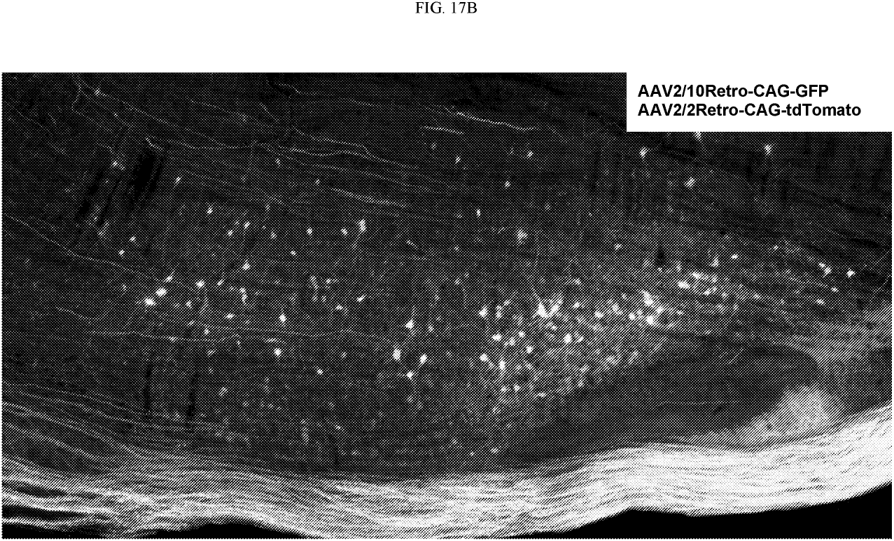

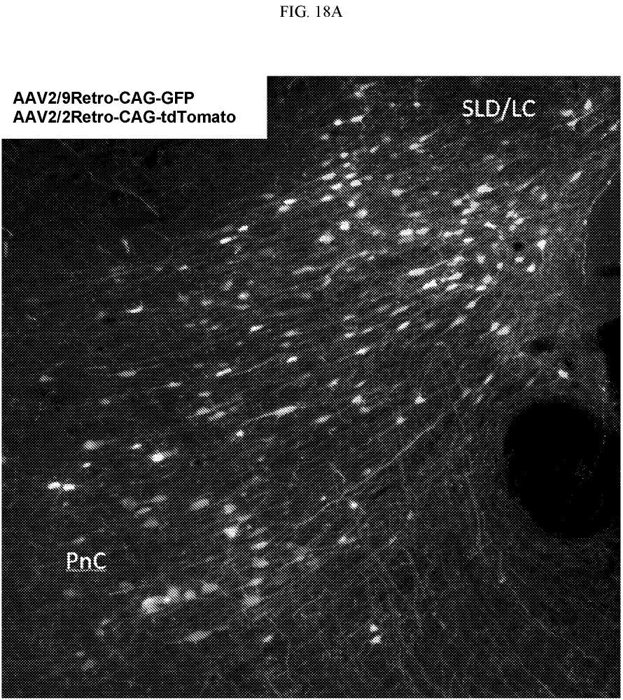

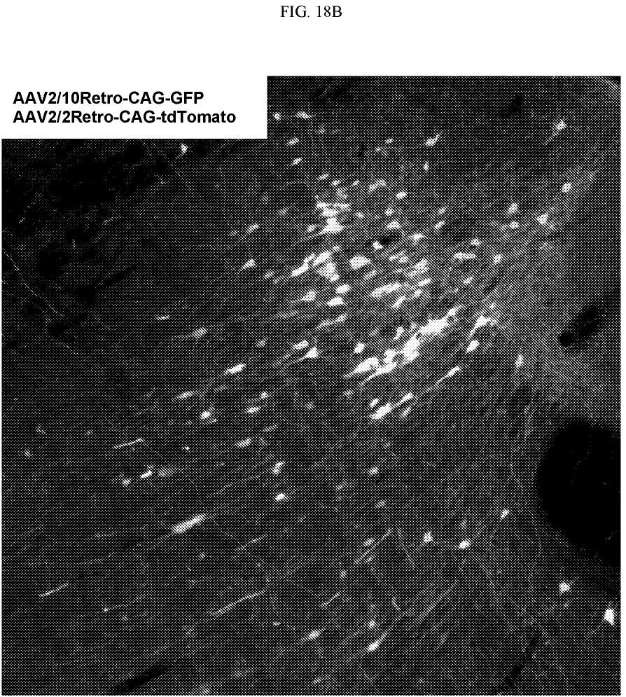

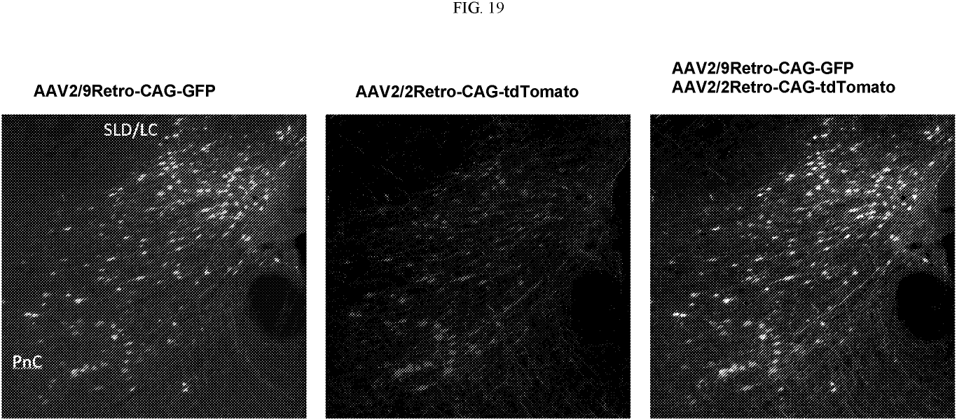

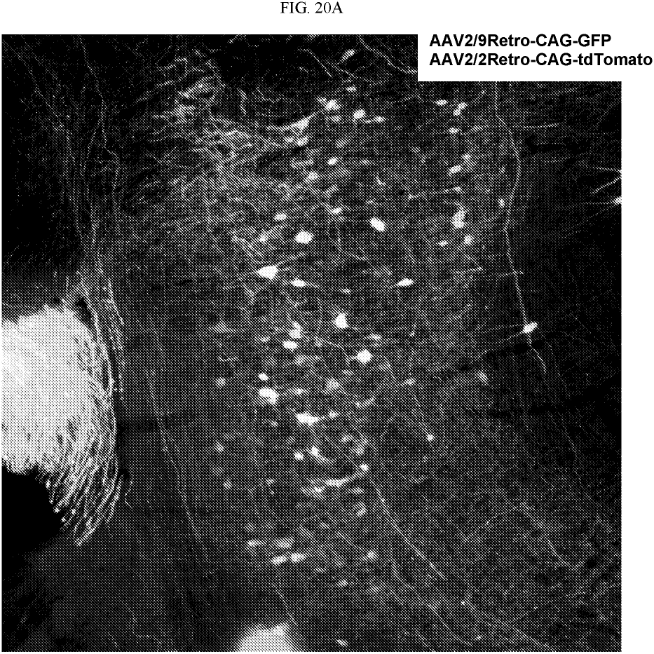

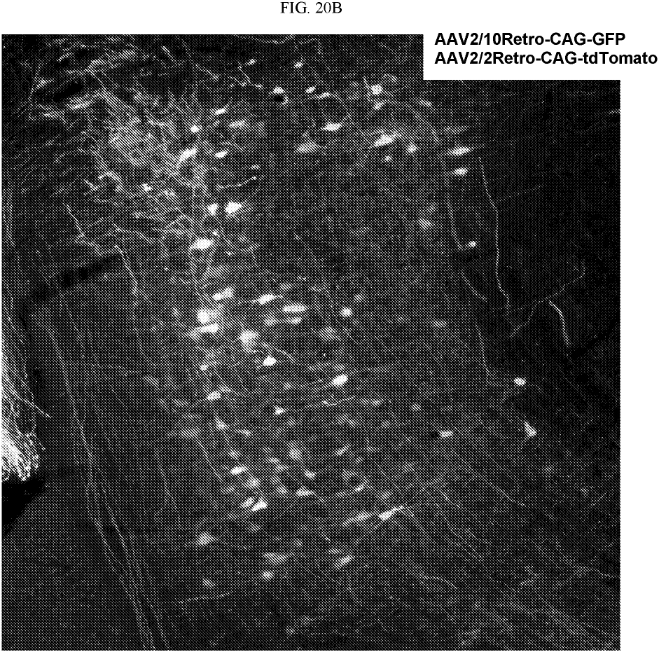

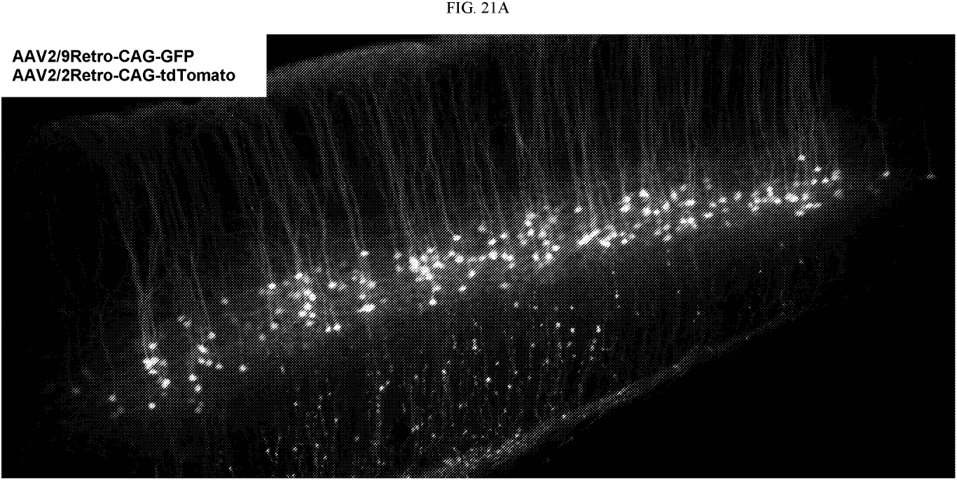

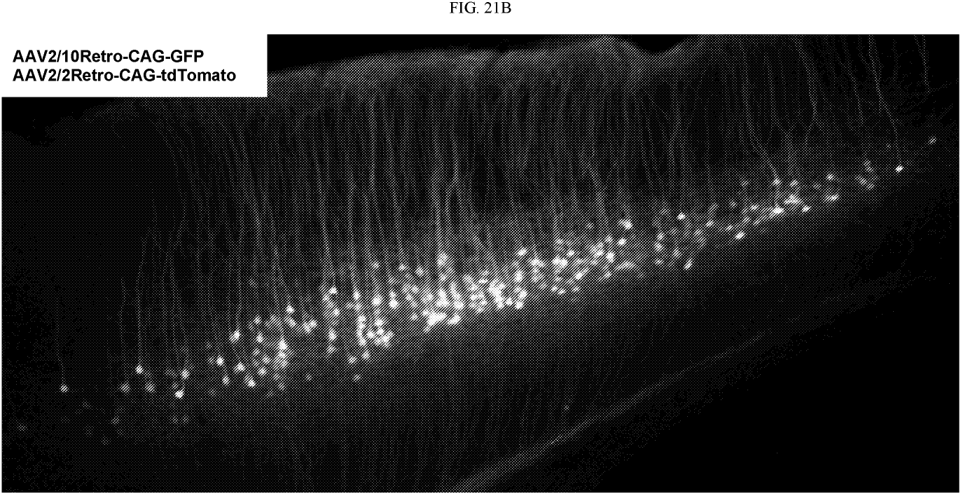

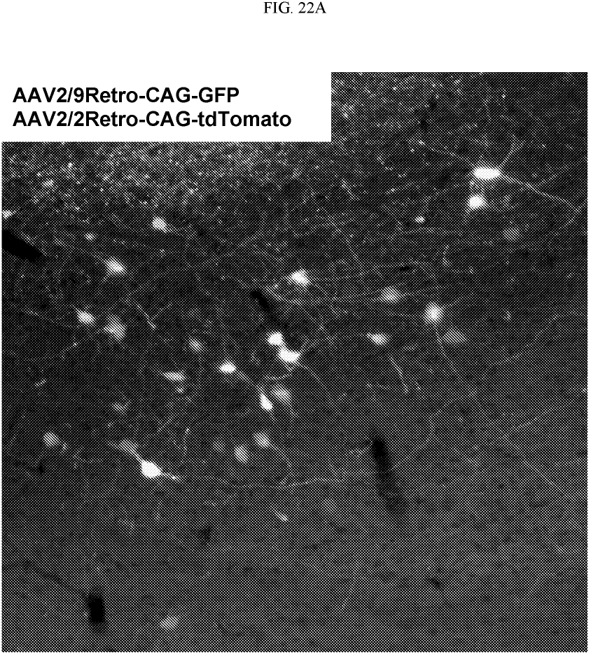

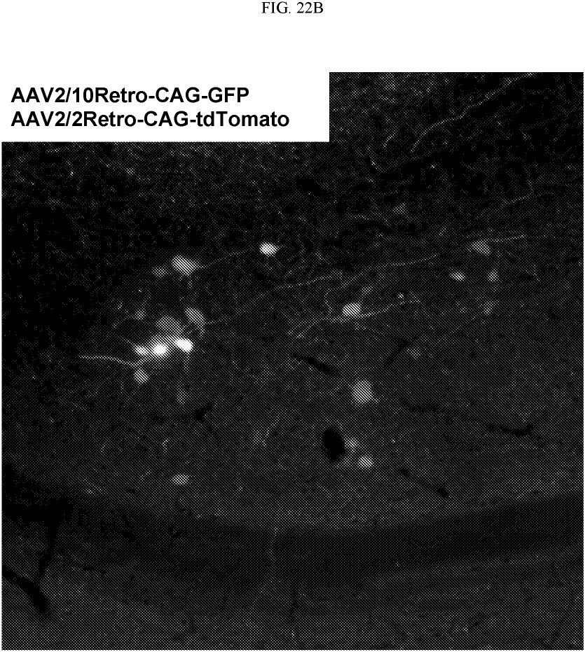

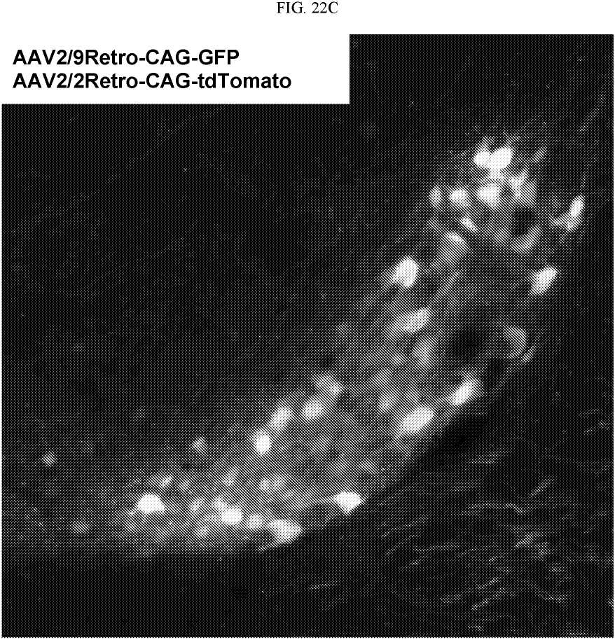

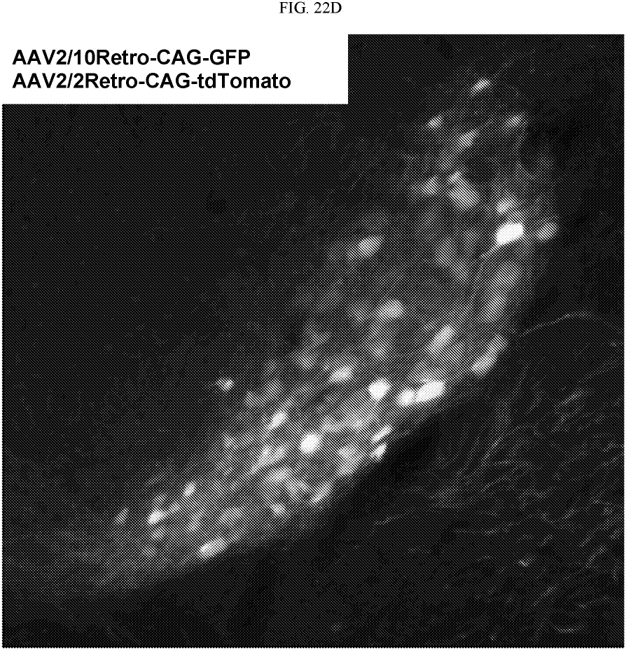

A comparison of AAV2/9Retro and AAV2/retro was carried out. AAV2/9Retro-CAG-GFP and AAV2/retro-CAG-tdTomato (5E+12 gc/ml) were co-injected into the lumber spine at L2-L4 at 12 points. 200 nl of a 5E+12 gc/ml (genome copies/ml) was injected at each point 3 hours following T8 crush. Interestingly, this resulted in robust expression for 3 weeks in a variety of brain regions, including cortex, cerebellum, and brain stem (FIG. 12). Expression of the reporters was observed at the injection site (FIGS. 13 and 14). Retrograde labeled cell bodies were observed in the gigantocellular reticular nucleus (Gi) (FIGS. 15A, 15B, 16, 17A, 17B). Retrograde labeled neurons were also observed in the sublaterodorsal tegmental nucleus (SLD) and locus coeruleus (LC) (FIGS. 18A, 18B, and 19), in the caudal pontine reticular nucleus (PnC) (FIGS. 18A, 18B, and FIG. 19), in the pontine reticular formation (PnO)(FIGS. 20A, 20B), and in the cortex (FIGS. 21A, 21B), and sub-cortex (FIGS. 22A-22D). These results demonstrate that intraspinal injection of the AAV2/9 vector led to better retrograde targeting than the AAV2/2Retro vector.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for targeted retrograde infection of an injured neuron, the method comprising administering a viral particle or virus-like particle to a subject with the injured neuron, thereby infecting the neuron, wherein the viral particle or virus-like particle comprises a virion protein 1 (VP1) polypeptide selected from the group consisting of AAV9Retro VP1 and AAV10Retro VP1, and wherein the polypeptide comprises the following amino acid sequence: LAxxDxTKxA (SEQ ID NO: 1) or LAxDxTKxxA (SEQ ID NO: 2), wherein X is any amino acid or is absent.

2. A method for transduction of a neuron proximal to a site of injury, the method comprising contacting the neuron with a viral particle or virus-like particle, thereby retrogradely infecting the neuron, wherein the viral particle or virus-like particle comprises a virion protein 1 (VP1) polypeptide selected from the group consisting of AAV9Retro VP1 and AAV10Retro VP1, and wherein the polypeptide comprises the following amino acid sequence: LAxxDxTKxA (SEQ ID NO: 1) or LAxDxTKxxA (SEQ ID NO: 2), wherein X is any amino acid or is absent.

3. A method for treatment of a neuron affected by an injury, disease, or disorder in a subject, the method comprising contacting the neuron with an effective amount of a viral particle or virus-like particle, wherein the viral particle or virus-like particle comprises a virion protein 1 (VP1) polypeptide selected from the group consisting of AAV9Retro VP1 and AAV10Retro VP1, and wherein the polypeptide comprises the following amino acid sequence: LAxxDxTKxA (SEQ ID NO: 1) or LAxDxTKxxA (SEQ ID NO: 2), wherein X is any amino acid or is absent.

4. The method of claim 1, wherein the VP1 is AAV9Retro VP1 or AAV10Retro VP1.

5. The method of claim 1, wherein the injury is caused by a traumatic insult.

6. The method of claim 1, wherein the injury is a spinal cord injury or traumatic brain injury.

7. The method of claim 1, wherein the neuron comprises an axon or cell body proximal to an injury.

8. The method of claim 1, wherein the disease or disorder is a neurodegenerative disease or disorder.

9. The method of claim 1, wherein the neuron is contacted within 7 days of the injury.

10. The method of claim 1, wherein the cell body of the neuron is in the gigantocellular reticular nucleus (Gi), the sublaterodorsal tegmental nucleus (SLD), the locus coeruleus (LC), the caudal pontine reticular nucleus (PnC), the pontine reticular formation (PnO), the cortex, the hypothalamic nuclei, or the red nucleus.

11. The method of claim 1, wherein the virus particle or virus-like particle encapsidates a heterologous polynucleotide sequence.

12. The method of claim 11, wherein the heterologous polynucleotide sequence encodes a polypeptide selected from the group consisting of growth factors, fluorescent proteins, phosphatase and tensin homolog (PTEN), suppressor of cytokine signaling 3 (SOCS3), or osteopontin (OPN).

13. The method of claim 12, wherein the growth factor is insulin-like growth factor 1 (IGF1).

14. An expression vector comprising a replication open reading frame from adeno-associated virus serotype 2 (AAV2 Rep), and a capsid open reading frame, wherein the capsid open reading frame is selected from the group consisting of AAV9Retro Cap and AAV10Retro Cap, and wherein the capsid open reading frame encodes a virion protein 1(VP1) comprising the following amino acid sequence: LAxxDxTKxA (SEQ ID NO: 1) or LAxDxTKxxA (SEQ ID NO: 2), wherein X is any amino acid or is absent.

15. The expression vector of claim 14, wherein the VP1 comprises the following amino acid sequence: LADQDYTKTA (SEQ ID NO: 3) and the capsid open reading frame is AAV9Retro Cap or AAV10Retro Cap.

16. A method for producing a viral particle or virus-like particle, the method comprising expressing in a cell or in vitro a replication open reading frame from adeno-associated virus serotype 2 (AAV2 Rep), and a capsid open reading frame, wherein the capsid open reading frame is selected from the group consisting of AAV9Retro Cap and AAV10Retro Cap, and wherein the capsid open reading frame encodes a virion protein 1 (VP1) comprising the following amino acid sequence: LAxxDxTKxA (SEQ ID NO: 1) or LAxDxTKxxA (SEQ ID NO: 2), wherein X is any amino acid or is absent.

17. A viral particle or virus-like particle produced by the method of claim 16.

18. A cell comprising the expression vector of claim 14.

19. A composition comprising the expression vector of claim 14.

20. A kit for transduction of an injured neuron, the kit comprising the viral particle or virus-like particle of claim 17.

21. A method for imaging an injured neuron, the method comprising:
(a) contacting the neuron with an effective amount of a viral particle or virus-like particle, wherein the viral particle or virus-like particle comprises a virion protein 1 (VP1) polypeptide selected from the group consisting of AAV9Retro VP1 and AAV10Retro VP1; wherein the VP1 polypeptide comprises the following amino acid sequence: LAxxDxTKxA (SEQ ID NO: 1) or LAxDxTKxxA (SEQ ID NO: 2), wherein X is any amino acid or is absent; and wherein the viral particle or virus-like particle encapsidates a polynucleotide encoding a fluorescent protein; thereby infecting the neuron and expressing the fluorescent protein in the neuron;
(b) fluorescently imaging the fluorescent protein in the neuron.
